(19) United States
(12) Patent Application Publication
(10) Pub. No.: US 2003/0166036 A1
(43) Pub. Date: Sep. 4, 2003

(54) Protease and an aminopeptidase associated with development of benign prostatic hyperplasia (BPH)

(76) Inventors: Stephen D. Mikolajczyk, San Diego, CA (US); Harry G. Rittenhouse, Del Mar, CA (US)

Correspondence Address: Wei- Ning Yang, Hogan & Hartson L.L.P., 500 south grand Avenue, Suite 1900, Los angeles, CA, 90071, US

(21) Appl. No.: 09/860,739

(22) Filed: May 18, 2001

(51) Int. Cl. C12Q 1/37; C12N 9/64

(52) U.S. Cl. 435/23; 435/226

(57) ABSTRACT

Methods for identification and isolation of a benign prostate specific antigen (BPSA) protease and BPSA aminopeptidase are provided. The BPSA protease of the present invention preferentially cleaves prostate specific antigen (PSA) at residue Lys182 and does not react with the residue Arg85. The BPSA aminopeptidase cleaves BPSA between residues Ile1 and Val2 and between residues Lys146 and Leu147, but does not cleave BPSA at Ser183.

### BACKGROUND OF THE ART

1. Area of the Art

The invention relates to proteases and aminopeptidases, and specifically to a protease and an aminopeptidase associated with the formation of hyperplastic prostate tissues, and the development of BPH nodules and methods of identification, isolation and use of the protease and the aminopeptidase. The invention is also related to inhibitors of the protease, and the aminopeptidase and the use of such inhibitors.

2. Description of the Prior Art

Throughout this application, various references are referred to within parentheses. Disclosures of these publications in their entireties are hereby incorporated by reference into this application to more fully describe the state of the art to which this invention pertains. Full bibliographic citations for these references may be found at the end of this application, preceding the claims.

Adenocarcinoma of the prostate accounts for a significant number of malignancies in men over 50. Currently used screening methods measure the serum level of prostate specific antigen (PSA) as a marker for the presence of prostate cancer (1-3). PSA is a 33 kD glycoprotein synthesized in the epithelial cells of the prostate gland. It is a secreted serine protease of the kallikrein family (extensively reviewed in (4)). The mature form of PSA has isoleucine as the N-terminal and 237 amino acid residues with a molecular mass of 28,400 D (5,6).

While PSA-based assays are useful in the diagnosis of prostate disease, they are not sufficiently specific to distinguish between benign prostate hyperplasmia (BPH) and prostate cancer (PCa). Several different approaches have been taken to improve the specificity of PSA-based assays. For example, recently, elevated levels of inactive and non-complexed with α₁-antichymotrypsin (ACT) forms of PSA (free PSA) in the serum of men with prostate cancer have been correlated with benign prostatic disease (7,8). Since 70 to 95% of serum PSA is normally complexed and the remaining PSA is free PSA (8-11), by evaluating a ratio of free PSA to total PSA, a substantial improvement in the diagnostic discrimination between BPH and PCa may be achieved.

In one of the earliest studies on free PSA isolated from seminal plasma, internal cleavage sites at Arg85, Lys145, and Lys182 (mistakenly identified as Lys185) were observed (12). Subsequent studies have focused largely on the predominant cleavage site at Lys145 (present in 30-40% of the PSA). The presence of Lys145 cleavage was correlated with inactivation of PSA and attributed to a random physiological cleavage, which occurs some time after PSA expression. The minor levels of cleavages at Arg85 and Lys182 have been observed but largely ignored (12,13).

PSA has also been isolated from BPH tissue nodules in order to determine whether this form of PSA was different from seminal plasma PSA. BPH nodules are comprised of a mixture of stromal elements and tightly packed epithelial ductal cells, and are visible by either macroscopic examination or low power microscopy of stained prostate tissue sections (14-16). The development of BPH nodules is highly correlated with increased prostate volume. The biochemical changes associated with nodular development may therefore play a role in the overall enlargement of the prostate, and in the clinical symptoms associated with BPH. PSA from BPH nodules has been found to contain a higher percentage of internal cleavages at Ile1, His54, Phe57, and Lys146 than seminal plasma PSA. These cleavages are thought to account for the lower enzymatic activity of PSA from BPH nodules as compared to that of seminal plasma PSA (17).

The U.S. patent application Ser. No. 09/303,208, filed on Apr. 30, 1999, entitled “Novel Forms of Prostate Specific Antigen (PSA) Specific for Benign Prostatic Hyperplasia (BPH) and Methods of Using Such,” which has been commonly assigned to the assignee of the present invention and is incorporated by reference herein, discloses a specific molecular form of PSA, benign PSA (BPSA), which is highly correlated with nodular development in the prostate transitional zone of patients with BPH. BPSA is characterized by two internal peptide bond cleavages at Lys182 and Lys145, but may also include additional one or more cleavages at Ile1, Lys145, and Lys146. BPSA has distinct chromatographic properties and can be separated from a mature form of PSA or other forms of PSA by high performance hydrophobic interaction chromatography (HIC-HPLC). BPSA is inactive (lacks chymotrypsin-like enzymatic activity) and, therefore, is present in serum as free PSA, not as PSA-ACT complex.

While the Lys182 cleavage of PSA has been observed in previous studies, no relevance was attached to this cleavage site and it was frequently ignored as a minor and insignificant cleavage site compared to Lys145. By contrast, the cleavage site at Lys145 has been the subject of much investigation because of its high percentage in seminal plasma PSA. It has been speculated that PSA cleaved at Lys145 could represent the major portion of the free PSA in the serum of patients with benign prostate disease. It follows from the high prevalence of the Lys145 cleavage in seminal plasma PSA that this is the most susceptible trypsin-like cleavage site in PSA. It has been assumed that the cleavage at the other trypsin-like sites, Arg85 and Lys182, reflects a less labile peptide sequence or decreased steric availability at these sites.

Due to a strong correlation between elevated levels of BPSA in hyperplastic prostate tissues and the presence of BPH nodules, a need exists for identification and characterization of proteases responsible for the formation of BPSA.

### SUMMARY OF THE INVENTION

While BPSA itself has not been shown to have biological or physiological significance beyond its role as a biochemical marker for the presence of BPH nodules in hyperplastic tissues, the protease responsible for the formation of BPSA could be physiologically important. For one, the BPSA protease may be involved in the biochemical pathway for the development of hyperplasia and BPH nodule formation. As seen with other extracellular matrix proteases, such as urokinase, which are involved in the cancer growth and metastasis (18,19), it is likely that BPSA proteases are involved in the tissue rearrangement characteristic of hyperplastic cell growth and BPH nodular formation. Accordingly, BPSA proteases may be used as therapeutic targets in BPH treatment. Similarly, other proteases with specificity to BPSA may play an important role in development of hyperplasia and BPH nodules and may be beneficial therapeutic targets as well.

Therefore, it is an object of the present invention to provide a method for the identification and isolation of a protease capable of cleaving PSA to form BPSA. Another object of the present invention is to identify and isolate proteases capable of further cleaving BPSA.

These and other objects are achieved in the present invention by a study of major internal cleavage sites of free PSA and particularly BPSA. While three major internal cleavage cites are present in the previously described PSA (Arg85, Lys145, and Lys182), unexpectedly, there is no significant cleavage of BPSA at Arg85 residue. This finding suggests that the protease responsible for the formation of BPSA (BPSA protease) has a higher specificity for the lysine residues. It is another unexpected discovery of the present invention that Lys182 is cleaved approximately 30 times more rapidly than Lys145 by BPSA protease.

Accordingly, one aspect of the present invention provides a method for identification and isolation of a benign prostate specific antigen (BPSA) protease. The method comprises:

(a) providing an extract from a source with elevated BPSA;

(b) screening the extract for a BPSA protease that is capable of cleaving PSA of SEQ ID NO:1 at residue Lys182 but not at residue Arg85; and

(c) isolating the BPSA protease from the extract if it is present.

The present invention further discloses a surprise correlation between the presence of the cleavage at Lys182 and the presence of the cleavages after Ile1 and Lys146 in BPSA. These later cleavages result in two novel peptides with N-termini beginning at Val2 (SEQ ID NO:2) and at Leu147 (SEQ ID NO:3). The presence of two simultaneous and correlated cleavages that remove a single N-terminal amino acid strongly suggests the presence of an aminopeptidase in the BPH nodules that is not normally present in other prostate tissues. While these two N-terminal cleavages have been observed previously in the PSA isolated from BPH nodules, it is an unexpected discovery of the present invention that the levels of these cleavage sites are correlated with each other and with the cleavage at Lys182. Accordingly, the discovered aminopeptidase (BPSA aminopeptidase) is probably expressed in a manner similar to the expression of BPSA protease. Since there is no evidence of a similarly modified amino-terminus at the Ser183 site, the discovered BPSA aminopeptidase does not have specificity for the Ser183 amino-terminus.

Accordingly, another aspect of the present invention provides a method for the identification and isolation of the BPSA aminopeptidase. The method comprises:

(a) providing an extract from a source with elevated BPSA;

(b) screening the extract for a BPSA aminopeptidase that is capable of cleaving BPSA between residues Ile1 and Val2, and between residues Lys146 and Leu147; and

(c) isolating the BPSA aminopeptidase from the extract if it is present.

The isolated BPSA protease and BPSA aminopeptidase are plausible targets for the treatment of BPH. It is possible that the inhibition of these enzymes will result in the reduction of BPH symptoms. Therefore, another aspect of the present invention encompasses inhibitors of the BPSA protease and BPSA aminopeptidase.

To ensure the efficacy of BPH therapy targeted to inhibit the expression and/or activity of BPSA protease and BPSA aminopeptidase, the levels of these enzymes and their activity must be closely monitored. Accordingly, this invention provides a method for detecting the presence of a BPSA protease in a sample. The method comprises the steps of:

(a) providing a sample suspected of containing the BPSA protease;

(b) contacting the sample with PSA under conditions sufficient to allow cleavage of PSA by the BPSA protease to form BPSA; and

(c) detecting the presence of BPSA, wherein the presence of BPSA indicates the presence of the BPSA protease.

Another aspect of the present invention provides a method for detecting the presence of a BPSA aminopeptidase in a sample. The method comprises the steps of:

(a) providing a sample suspected of containing the BPSA aminopeptidase;

(b) contacting the sample with BPSA under conditions sufficient to allow cleavage of BPSA by the BPSA aminopeptidase to cleave BPSA between residues Ile1 and Val2, and between residues Lys146 and Leu147 to produce peptides of SEQ ID NO:2 and SEQ ID NO:3; and

(c) detecting the presence of the peptides of SEQ ID NO:2 and SEQ ID NO:3 wherein the presence of said peptides indicates the presence of the BPSA aminopeptidase.

The invention also provides a DNA molecule comprising a nucleotide sequence encoding the proteases. A transformant host cell transformed with the replicable expression vehicle encoding the protease of the invention, which expression vehicle may be either prokaryotic or eukaryotic, also forms part of the present invention. The BPSA protease and BPSA aminopeptidase in accordance with the invention may be produced recormbinantly by culturing a transformant host cell of the invention in a suitable culture medium and isolating the protease.

BPSA protease and BPSA aminopeptidase of the present invention, as well as variants and subunits thereof, can be used to produce populations of antibodies that are specific for BPSA protease and the aminopeptidase. Thus, another aspect of the present invention provides an antibody, preferably a monoclonal antibody, which specifically binds to BPSA protease and an antibody which specifically binds to BPSA aminopeptidase.

The present invention also encompasses a method for detecting BPSA protease and BPSA aminopeptidase in a sample by utilizing the antibodies of the present invention. Samples may include seminal fluid, blood or serum; tissues, such as prostate carcinomas; or cells, such as prostate cells. Direct and competitive assays to detect BPSA protease and BPSA aminopeptidase are also included within the scope of the present invention.

Based on the discoveries of the present invention, one aspect of the present invention provides diagnostic methods for detecting benign prostatic hyperplasia (BPH) in a subject. In accordance with embodiments of the present invention, such a method includes the steps of determining the amount and/or activity of BPSA protease and BPSA aminopeptidase contained in a sample obtained from the subject, and correlating the amount and/or activity of the enzymes to the presence of BPH in the subject. The presence of these proteases may influence biochemical pathways in the formation of hyperplastic tissues and nodule formation, which are the pathological features of BPH.

Kits for detecting benign prostatic hyperplasia are also included as embodiments of the present invention. Pharmaceutical compositions comprising an inhibitor of the protease of the invention as the active ingredient together with a pharmaceutically acceptable carrier form yet another aspect of the invention. If the BPH protease is involved in the biochemical pathway leading to hyperplasia and nodule formation, then this protease becomes a target for therapeutic intervention. This is similar in concept to drugs that inhibit the 5-alpha-reductase enzyme and the androgen pathway in prostate tissues as a treatment for BPH (20).

The invention is defined in its fullest scope in the appended claims and is described below in its preferred embodiments.

### DESCRIPTION OF THE FIGURES

The above-mentioned and other features of this invention and the manner of obtaining them will become more apparent, and will be best understood, by reference to the following description, taken in conjunction with the accompanying drawings. These drawings depict only a typical embodiment of the invention and do not therefore limit its scope.

FIG. 1 is an HIC-HPLC chromatogram overlay showing PSA before (Peak A) and after (Peaks B and C) incubation with LysC for 30 minutes. Peak B represents PSA cleaved at Lys182, and Peak C represents PSA cleaved at both Lys182 and Lys145.

FIG. 2 is an SDS-PAGE profile of the 3 peaks seen in FIG. 1.

FIG. 3 shows the profile of the PSA peaks obtained after incubation with LysC at different times. Peak A is starting PSA; Peak B is PSA cleaved at Lys182; Peak C is PSA cleaved at Lys182 and Lys145.

FIG. 4 demonstrates the relative percentage of the PSA cleaved at Lys182 compared to Lys145 at different times after incubation with LysC. Values were obtained from the peak areas calculated from HIC-HPLC, as seen in FIG. 3.

FIG. 5 is a cartoon depicting the order of internal PSA cleavage sites obtained after incubation with LysC.

FIG. 6 is the profile of the proPSA peaks obtained after incubation with LysC at different times. Peak A is starting proPSA; Peak B is proPSA cleaved at Lys182; Peak C is proPSA cleaved at Lys182 and Lys145.

FIG. 7 shows the relative percentage of the proPSA cleaved at Lys182 compared to Lys145 at different times after incubation with LysC. Values were obtained from the peak areas calculated from HIC-HPLC, as seen in FIG. 6.

FIG. 8 is a stacked line graph of the Lys182 and residual Lys145 cleavage sites in the PSA purified from six different specimens of transurethral resection of prostate (TLRP) tissue. Residual %145 is the percentage of PSA cleaved at Lys145 that is not associated with BPSA. %182 is the percentage of PSA cleaved at Lys182.

FIG. 9 shows an HIC-HPLC chromatogram overlay of the Lys182-cleaved PSA purified from TURP tissue containing low (TURP-1) and high (TURP-6) levels of BPSA, as seen in FIG. 8. The Lys182-cleaved forms of PSA were purified from the other forms of PSA using PS2E290 mAb. Peak A is the BPSA which is cleaved at both Lys182 and Lys145. Peak B contains PSA cleaved only at Lys182.

FIG. 10 shows the HIC-HPLC chromatogram profile of the total PSA purified from TURP-2.

FIG. 11 is the stacked histogram showing the relative percentage of the 4 major cleavage sites in 6 TURP tissue samples. Samples are arranged in increasing %Lys182 cleavage.

FIG. 12 is a stacked histogram showing the primary BPSA cleavage sites, Ile1, Lys146, and Lys182, present in PSA purified from individual seminal plasma specimens.

### DETAILED DESCRIPTION OF THE INVENTION

Recently, elevated levels of benign prostate specific antigen (BPSA) in tissues and seminal plasma have been correlated with benign prostatic hyperplasia (BPH) (21,22). BPSA is characterized by the post-translational peptide bond cleavage at Lys145 and Lys182 by unknown proteases. Accordingly, proteases responsible for BPSA formation are likely to be involved in the biochemical pathway for benign hyperplastic cell growth and BPH nodule formation.

It is an unexpected discovery of the present invention that PSA identified in samples from BPH patients is cleaved at Lys182 but not at residue Arg85. In one embodiment, PSA isolated from BPH patients are additionally cleaved at Lys145. It is also an unexpected discovery that protease responsible for BPSA production has preferred specificity toward the Lys182 site as compared to the Lys145 site. The Lys182 and Lys145 cleavages cause BPSA to adopt an altered conformation as compared to PSA that is recognized by HIC-HPLC.

In a non-nodular environment, the cleavage at Lys145 predominates both in tissues and in seminal plasma, and this PSA form is not distinguishable from non-cleaved PSA by HIC-HPLC (22). BPH nodules and hyperplastic tissues, therefore, contain either a newly expressed or significantly increased expression of BPSA protease that has a preference for the Lys182 site. Accordingly, BPSA protease is a plausible target for therapeutic strategies since proteases are very likely to be involved in the process of hyperplastic tissue growth and tissue matrix rearrangement.

Accordingly, one aspect of the present invention provides a method for identification and isolation of a benign prostate specific antigen (BPSA) protease. The method comprises:

(a) providing an extract from a source with elevated BPSA;

(b) screening the extract for a BPSA protease that is capable of cleaving PSA of SEQ ID NO:1 at residue Lys182 but not at residue Arg85; and

(c) isolating the BPSA protease from the extract if it is present.

For the purposes of the present invention, any sources with elevated BPSA may be used for the purpose of isolating a BPSA protease. Examples of such a source include, but are not limited to, BPH tissues, cells, extracts, and seminal plasma. Established prostate cell lines can also be utilized. In general, any prostate cells expressing PSA can be employed as the source for the protease, though hyperplastic prostate tissues would be the preferred source.

The preparation of extracts from sources with elevated BPSA can be effected by methods known in the art. Examples of such a method include, but not limited to, homogenization or sonication of cells in a buffered solution in order to release the cellular contents.

The term “BPSA”, as used herein, refers to a form of PSA that comprises at least one cleavage at Lys182 of the amino acid sequence of a mature form of PSA. A mature form of PSA has 237 amino acid residues with a molecular mass of 28,400 D and the amino acid sequence identified by SEQ ID NO:1. The BPSA of the present invention has the same amino acid sequence of a mature form of PSA, except that the polypeptide chain of the PSA of the present invention has been hydrolyzed between residues 182 and 183. In accordance with embodiments of the present invention, a BPSA of the present invention may also include additional one or more cleavages at Ile1, Lys145, and Lys146 of the amino acid sequence of a mature PSA.

For the purpose of the present invention, the term “cleavage” means the hydrolysis of the peptide bond between the indicated amino acid and the amino acid C-terminal to that amino acid.

The BPSA of the present invention exists at an elevated level in the transition zone (TZ) of BPH tissue. The prostate is composed of three zones: the central zone, the peripheral zone (PZ), and the transition zone (TZ). The PZ comprises about 70% of the volume of a normal prostate, while the central zone and TZ are about 25% and 5%, respectively. All three zones are well defined in the art (23). Briefly, the TZ is characterized by small, simple glands embedded in a compact stroma, whereas the PZ is characterized by small glands embedded in a loose stroma. The TZ tissue forms a distinctive boundary with the PZ. The PZ and TZ are the zones of primary interest, since cancer is localized mostly in the PZ, while BPH is the result of tissue enlargement in the TZ. With extensive BPH, the TZ grows to several times the volume of other prostate zones. The TZ tissue surrounds the proximal prostate urethra, which is the reason that restricted urinary flow is a symptom of enlarged TZ resulting from BPH. It is worth noting that while restricted urinary flow can be caused by the increase in size of the transition zone and thus the total prostate volume, urinary flow symptoms do not correlate simply with transition zone size or total prostate volume. This paradox is yet another reason why understanding the underlying disease process and establishing specific markers of the disease are needed.

For the purpose of the present invention, the level of BPSA is elevated if the percentage of the BPSA compared to total PSA is higher than the percentage of the BPSA found in non-hyperplastic transition zone tissue. In accordance with one embodiment of the present invention, PSA extracted from BPH tissues contains from 5 to 30% of BPSA. The BPSA is lower or absent in the non-hyperplastic transition zone characterized by the absence of BPH nodules.

The BPSA is inactive, i.e., it lacks chymotrypsin-like enzymatic activity and therefore is present in serum as free PSA, not complexed to any protease inhibitors. The most common protease inhibitors that bind to active PSA are alpha-1-antichymotrypsin (ACT) and alpha-2-macroglobulin (A2M). PSA-ACT complex is detected by commercially available total PSA assays. PSA complexed with A2M is not detected by commercially available total PSA assays. For the purpose of the present invention, a free PSA is a PSA that is not complexed and cannot complex with any protease inhibitor.

The extract of the present invention is screened for a protease that is capable of cleaving PSA of SEQ ID NO:1 at residue Lys182 but not at residue Arg85. In general, a screening process includes the incubation of PSA, which does not contain the cleavage site at Lys182 with an extract solution, and testing the PSA for the presence of PSA cleaved at Lys182. In accordance with one embodiment of the present invention, the screening step may further include the steps of:

(a) contacting a sample from the extract with PSA under conditions sufficient to allow cleavage of PSA by the BPSA protease to form BPSA; and

(b) detecting the presence of BPSA, wherein the presence of BPSA indicates the presence of the BPSA protease.

For the purpose of the present invention, conditions are sufficient if they allow the binding of BPSA protease to PSA and the cleaving of PSA to form BPSA. For example, in one embodiment, 100 ug of protein from the extract is incubated with 1 ug of PSA for 1 hour at 37° C. in 100 ul of 100 mM Tris buffer, pH 8. The detection step may be readily carried out by any method that specifically recognizes BPSA in a sample. In one embodiment, for example, BPSA is detected by using labeled antibodies which specifically react with BPSA, as described in the above-referenced U.S. patent application Ser. No. 09/303,208 filed on Apr. 30, 1999.

The isolating step may be performed by utilizing any known methods for purifying proteins and polypeptides. For example, in one embodiment, a combination of size exclusion, anion exchange, and hydrophobic interaction chromatography could be used to isolate the BPSA protease. Preferably, the isolated and purified protease is further characterized by determining its molecular weight, physical and chemical properties, amino acid sequence, and specificity. These are standard procedures and are well known in the art.

In one embodiment, BPSA protease also cleaves PSA at residue Lys145. Preferably, BPSA protease preferentially cleaves at Lys182 over Lys145. For the purpose of the present invention, the term “preferentially cleaves” means that the rate of the cleavage at the Lys182 site is faster than the rate at the Lys145 site. Preferably, cleavage after Lys182 is at least ten times faster than the rate at the Lys145 site. In one embodiment, the rate of cleavage at Lys182 site is 30 times faster than at the Lys145 site.

It is another unexpected discovery of the present invention that Ile1 and Lys146 cleavages of BPSA strongly correlate with the cleavage at Lys182. These results were observed for both hyperplastic tissue and seminal plasma samples. The unexpected high levels of Ile1 and Lys146 cleavages in BPSA indicate the presence of an aminopeptidase in hyperplastic tissues (BPSA aminopeptidase).

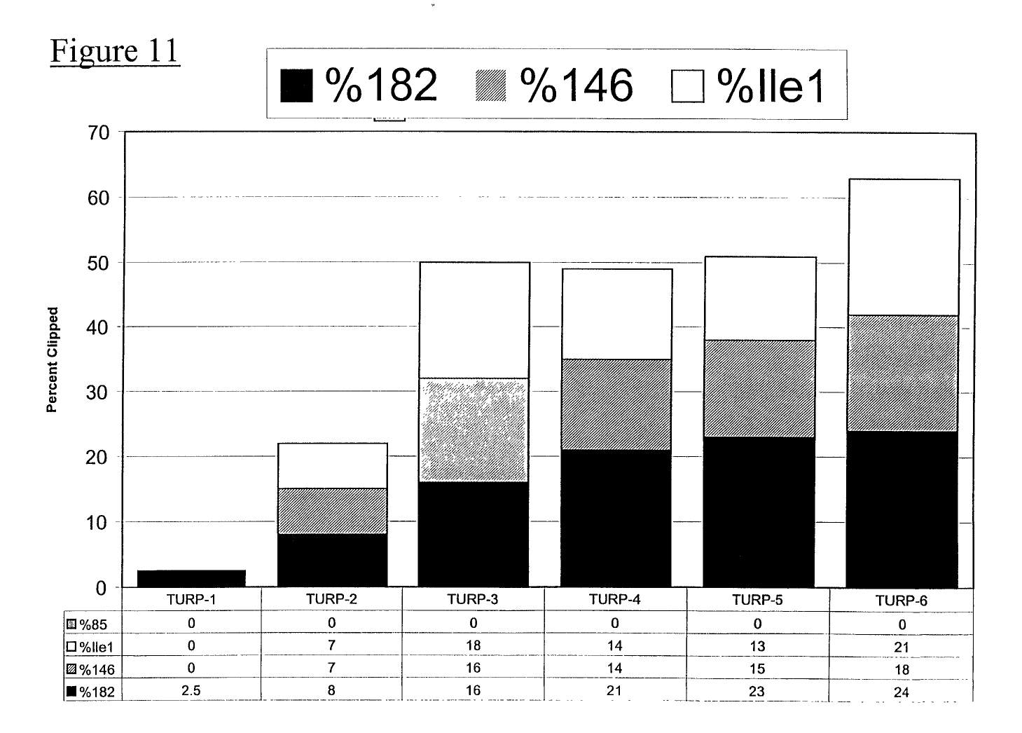

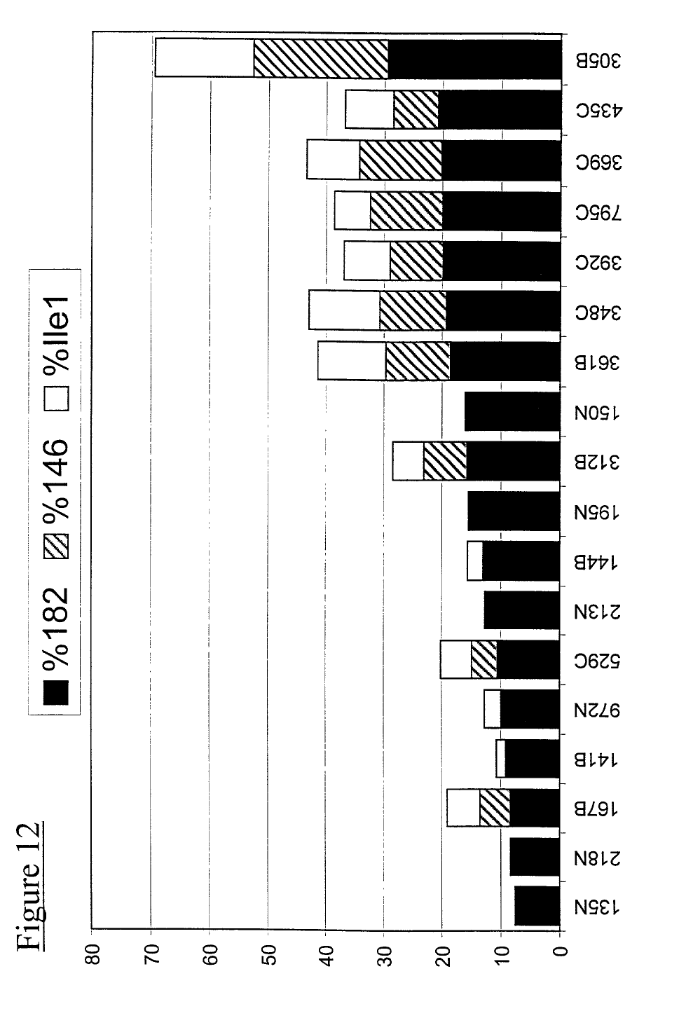

Similarly to the BPSA protease, BPSA aminopeptidase of the present invention may play an important role in the process of nodular development. The BPSA aminopeptidase appears to be either unique, or highly enriched in nodular tissue compared to other prostate tissues. The BPSA aminopeptidase is highly correlated with the BPSA protease as evidenced by the high correlation of the Ile1 and Lys146 cleavages in PSA from BPH nodules with the Lys182 cleavage in the PSA from both BPH tissues and seminal plasma (FIGS. 11 and 12). However, BPSA aminopeptidase has no activity toward the Lys182 N-terminus, as evidenced by the lack of a Thr184 N-terminus.

Accordingly, another aspect of the present invention provides a method for identification and isolation of a BPSA aminopeptidase. The method comprises:

(a) providing an extract from a source with elevated BPSA;

(b) screening the extract for a BPSA aminopeptidase that is capable of cleaving BPSA between residues Ile1 and Val2, and between residues Lys146 and Leu147; and

(c) isolating the BPSA aminopeptidase from the extract if it is present.

While other methods of screening may be used, in one embodiment, the screening step may include the steps of:

(a) contacting a sample from the extract with BPSA under conditions sufficient to allow cleavage of BPSA by the BPSA aminopeptidase to cleave BPSA between residues Ile1 and Val2, and between residues Lys146 and Leu147 to produce peptides of SEQ ID NO:2 and SEQ ID NO:3; and

(b) detecting the presence of the peptides of SEQ ID NO:2 and SEQ ID NO:3 wherein the presence of said peptides indicates the presence of the BPSA aminopeptidase.

For the purpose of the present invention, conditions are sufficient if they allow the binding of BPSA aminopeptidase to BPSA and the cleaving of BPSA to produce peptides of SEQ ID NO:2 and SEQ ID NO:3. For example, in one embodiment, 100 ug of protein from the extract may be incubated with 1 ug of BPSA for 1 hour at 37° C. in 100 ul of 100 mM Tris buffer, pH 8. The detection step may be readily carried out by any method that specifically recognizes peptides SEQ ID NO:2 and SEQ ID NO:3 in a sample. In one embodiment, for example, the polypeptides are detected by N-terminal sequencing after the cleaved BPSA has been purified from the incubation mixture by affinity chromatography using anti-PSA mAbs such as PSM773. Other detection methods that may be used include, but are not limited to, anion or cation exchange chromatography. These methods are well known in the art and will not be discussed here.

As discussed above, the isolating step may be performed by utilizing any known methods for purifying proteins polypeptides. For example, in one embodiment, the combination of size exclusion, anion exchange, and hydrophobic interaction chromatography could be used to isolate the BPSA aminopeptidase. Preferably, the isolated and purified aminopeptidase is further characterized by determining its molecular weight, physical and chemical properties, amino acid sequence, and specificity. These are standard procedures and are well known in the art (24).

The isolated BPSA protease and the BPSA aminopeptidase are plausible targets for the treatment of BPH. It is possible that the inhibition of these enzymes will result in the reduction of BPH symptoms. Therefore, another aspect of the present invention encompasses inhibitors of the BPSA protease and the BPSA aminopeptidase. Inhibitors of the present invention may be obtained by techniques involving chemical synthesis, combinatorial chemistry to screen for optimal inhibition characteristics, enzyme inhibition analysis, and clinical testing of the desired inhibitors. Multiple techniques are well known in the art, for example, as in the development of inhibitors of the HIV protease (25) and the inhibitor of 5-alpha-reductase for the treatment of BPH (26,27), and therefore would not be repeated herein in details.

To ensure the efficacy of BPH therapy targeted to inhibit expression and/or activity of BPSA protease and BPSA aminopeptidase, it may be important to monitor the levels of these enzymes. Accordingly, this invention provides a method of detecting the presence of the BPSA protease in a sample. The method comprises the steps of:

(a) providing a sample suspected of containing the BPSA protease;

(b) contacting the sample with PSA under conditions sufficient to allow cleavage of PSA by the BPSA protease to form BPSA; and

(c) detecting the presence of BPSA, wherein the presence of BPSA indicates the presence of the BPSA protease.

Another aspect of the present invention provides a method of detecting the presence of a BPSA aminopeptidase in a sample. The method comprises the steps of:

(a) providing a sample suspected of containing the BPSA aminopeptidase;

(b) contacting the sample with BPSA under conditions sufficient to allow cleavage of BPSA by the BPSA aminopeptidase to cleave BPSA between residues Ile1 and Val2, and between residues Lys146 and Leu147 to produce peptides of SEQ ID NO:2 and SEQ ID NO:3; and

(c) detecting the presence of the peptides of SEQ ID NO:2 and SEQ ID NO:3 wherein the presence of said peptides indicates the presence of the BPSA aminopeptidase.

In accordance with one embodiment of the present invention, BPSA generated in vitro by LysC may be used as the substrate to test for the presence of this amino peptidase (see Example 1). In vitro-produced BPSA has only cleavages at Lys145 and Lys182. Such an assay should generate the peptide of SEQ ID NO:2 with the N-termini beginning at Val2 and the peptide of SEQ ID NO:3 with the N-termini beginning at Leu147. The assay should not generate the peptide of SEQ ID NO:5 with the N-terminus beginning at Thr184.

BPSA protease and BPSA aminopeptidase themselves may become markers for the detection or evaluation of BPH, since these proteases may be involved in the biochemical pathways responsible for the hyperplasia and nodule formation. In addition, once isolated, and in accordance with an embodiment of the present invention, BPSA protease and BPSA aminopeptidase can be used to produce antibodies.

In accordance with the present invention, an antibody which consists essentially of pooled monoclonal antibodies with different epitopic specificities, as well as distinct monoclonal antibody preparations, is provided. Monoclonal antibodies against purified BPSA protease and BPSA aminopeptidase can be prepared using known hybridoma cell culture techniques, for example, as described by E. Harlow et al., Antibodies: A Laboratory Manual, Cold Spring Harbor Laboratory, 1988. In general, this method involves preparing an antibody-producing fused cell line, e.g., of primary spleen cells fused with a compatible continuous line of myeloma cells, and growing the fused cells either in mass culture or in an animal species from which the myeloma cell line used was derived or is compatible. Such antibodies offer many advantages in comparison to those produced by the inoculation of animals, as they are highly specific and sensitive and relatively “pure” immunochemically. Immunologically active fragments of antibodies are also within the scope of the present invention, e.g., the f(ab) fragment, as are partially humanized monoclonal antibodies.

If desired, polyclonal antibodies can be further purified, for example, by binding to an elution from a matrix to which a polypeptide, or a peptide to which the antibodies were raised, is bound. Those skilled in the art will know of various techniques common in the immunology arts for purification and/or concentration of polyclonal antibodies, as well as monoclonal antibodies. (See, for example, Coligan et al., Unit 9, Current Protocols in Immunology, Wiley Interscience, 1991, incorporated by reference.)

The term “antibody,” as used in this invention, includes intact molecules as well as fragments thereof, such as Fab, F(ab′)₂, and Fv, which are capable of binding the epitopic determinant. These antibody fragments retain some ability to selectively bind with their antigen or receptor.

Accordingly, one aspect of the present invention provides an antibody that specifically binds to BPSA protease and an antibody that specifically binds to BPSA aminopeptidase of the present invention. The term “specifically immunoreactive or specifically binds to,” as used herein, indicates that the antibodies of the present invention recognize and bind to antigenic determinants or epitopes that are unique to BPSA protease and BPSA aminopeptidase.

Antibodies of the present invention may be used for detecting and determining the presence and amount of BPSA protease and BPSA aminopeptidase in a sample. In accordance with the present invention, the BPSA protease and BPSA aminopeptidase may be detected in patient tissue samples by immunohistochemical methods and/or in patient fluid samples by in vitro immunoassay procedures.

Immunohistochemical methods for the detection of antigens in patient tissue specimens are well known in the art and need not be described in detail herein. For example, methods for the immunohistochemical detection of antigens are generally described in Taylor, Arch. Pathol. Lab. Med. Briefly, in the context of the present invention, a tissue specimen obtained from a patient suspected of having a prostate-related problem is contacted with an antibody, preferably a monoclonal antibody, recognizing BPSA protease or BPSA aminopeptidase. The site at which the antibody is bound is thereafter determined by selective staining of the tissue specimen by standard immunohistochemical procedures. In one embodiment of the present invention, the tissue specimen is a tissue specimen obtained from the prostate of a patient. The prostate tissue may be a normal or benign prostate tissue, a cancer prostate tissue, or a benign prostatic hyperplasia tissue.

Similarly, the general methods of the in vitro detection of antigenic substances in patient fluid samples by immunoassay procedures are also well known in the art and require no repetition herein. For example, immunoassay procedures are generally described in Paterson et al., Int. J. Can. 37:659 (1986) and Burchell et al., Int. J. Can. 34:763 (1984). According to one embodiment of the present invention, an immunoassay for detecting BPSA protease and BPSA aminopeptidase in a biological sample comprises the steps of: (a) contacting an antibody that specifically binds to BPSA protease (or BPSA aminopeptidase) with the sample under a condition that allows the formation of a binary complex comprising the BPSA protease (or BPSA aminopeptidase) and the antibody; and (b) detecting and determining the amount of the complex.

For the purpose of the present invention, the biological sample can be any human physiological fluid sample that contains BPSA protease and BPSA aminopeptidase of the present invention. Examples of the human physiological fluid sample include, but are not limited to, blood, serum, seminal fluid, urine, and plasma.

For the purpose of the present invention, both monoclonal antibodies and polyclonal antibodies may be used as long as such antibodies possess the requisite specificity for the antigen provided by the present invention. Preferably, monoclonal antibodies are used.

Monoclonal antibodies can be utilized in a liquid phase or bound to a solid phase carrier. Monoclonal antibodies can be bound to many different carriers and used to determine the BPSA protease or BPSA aminopeptidase of the present invention. Examples of well-known carriers include glass, polystyrene, polypropylene, polyethylene, dextran, nylon, amylases, natural and modified celluloses, polyacrylamides, agaroses, and magnetites. The nature of the carrier can be either soluble or insoluble for purposes of the invention. Examples of insoluble carriers include, but are not limited to, a bead and a microtiter plate. Those skilled in the art will know of other suitable carriers for binding monoclonal antibodies or will be able to ascertain such under routine experimentation.

In addition, the monoclonal antibodies in these immunoassays can be detectably labeled in various ways. For example, monoclonal antibodies of the present invention can be coupled to low molecular weight haptens. These haptens can then be specifically detected by means of a second reaction. For example, it is common to use such haptens as biotin, which reacts with avidin, or dinitrophenyl, pyridoxal and fluorescein, which can react with specific antihapten antibodies. In addition, monoclonal antibodies of the present invention can also be coupled with a detectable label, such as an enzyme, radioactive isotope, fluorescent compound or metal, chemiluminescent compound, or bioluminescent compound. Furthermore, the binding of these labels to the desired molecule can be done using standard techniques common to those of ordinary skill in the art.

One of the ways in which the antibody can be detectably labeled is by linking it to an enzyme. This enzyme, in turn, when later exposed to its substrate, will react with the substrate in such a manner as to produce a chemical moiety which can be detected by, for example, a spectrophotometric or fluorometric means (ELISA system). Examples of enzymes that can be used as detectable labels are horseradish peroxidase, malate dehydrogenase, staphylococcal nuclease, delta-5-steroid isomerase, yeast alcohol dehydrogenase, alpha-glycerophosphate dehydrogenase, triose phosphate isomerase, alkaline phosphatase, asparaginase, glucose oxidase, beta-galactosidase, ribonuclease, urease, catalase, glucose-6-phosphate dehydrogenase, glucoamylase, and acetylcholine esterase.

For increased sensitivity in the ELISA system, the procedures described can be modified using biotinylated antibodies reacting with avidin-peroxidase conjugates.

The amount of antigen can also be determined by labeling the antibody with a radioactive isotope. The presence of the radioactive isotope would then be determined by such means as the use of a gamma counter or a scintillation counter. Isotopes which are particularly useful are ³H, ¹²⁵I, ¹²³I, ³²P, ³⁵S, ¹⁴C, ⁵¹Cr, ³⁶Cl, ⁵⁷Co, ⁵⁸Co, ⁵⁹Fe, ⁷⁵Se, ¹¹¹N, ⁹⁹mTc, ⁶⁷Ga, and ⁹⁰Y.

The determination of the antigen is also possible by labeling the antibody with a fluorescent compound. When the fluorescently labeled molecule is exposed to light of the proper wave length, its presence can then be detected due to the fluorescence of the dye. Among the most important fluorescent-labeling compounds are fluorescein isothiocyanate, rhodamine, phycoerythrin, phycocyanin, allophycocyanin, o-phthaldehyde, and fluorescamine.

Fluorescence-emitting metal atoms, such as Eu (europium) and other lanthanides, can also be used. These can be attached to the desired molecule by means of metal-chelating groups, such as DTPA or EDTA.

Another way in which the antibody can be detectably labeled is by coupling it to a chemiluminescent compound. The presence of the chemiluminescent-tagged immunoglobulin is then determined by detecting the presence of luminescence that arises during the course of a chemical reaction. Examples of particularly useful chemiluminescent labeling compounds are luminol, isoluminol, aromatic acridinium ester, imidazole, acridinium salt, and oxalate ester.

Likewise, a bioluminescent compound may also be used as a label. Bioluminescence is a special type of chemiluminescence which is found in biological systems in which a catalytic protein increases the efficiency of the chemiluminescent reaction. The presence of a bioluminescent molecule would be determined by detecting the presence of luminescence. Important bioluminescent compounds for purposes of labeling are luciferin, luciferase, and aequorin.

Qualitative and/or quantitative determinations of BPSA protease and BPSA aminopeptidase of the present invention in a sample may be accomplished by competitive or non-competitive immunoassay procedures in either a direct or indirect format. Examples of such immunoassays are the radioimmunoassay (RIA) and the sandwich (immunometric) assay. The detection of the antigens using the monoclonal antibodies of the present invention can be done utilizing immunoassays which are run in either the forward, reverse, or simultaneous modes, including immunohistochemical assays on physiological samples. Those skilled in the art will know, or can readily discern, other immunoassay formats without undue experimentation.

The terms “immunometric assay” or “sandwich immunoassay” include a simultaneous sandwich, forward sandwich, and reverse sandwich immunoassay. These terms are well understood by those skilled in the art. Those skilled in the art will also appreciate that antibodies according to the present invention will be useful in other variations and forms of assays which are presently known or which may be developed in the future. These are intended to be included within the scope of the present invention.

A useful immunoassay which can be practiced in accordance with an embodiment of the present invention is the two-antibody sandwich technique. These assays are used primarily to determine the antigen concentration in unknown samples. Two-antibody assays are quick and accurate, and if a source of pure antigen (in this case, BPSA protease or BPSA aminopeptidase) is available, the assays can be used to determine the absolute amounts of antigen in unknown samples. The assay requires two antibodies that bind to non-overlapping epitopes on the antigen. Either two monoclonal antibodies that recognize discrete sites or one batch of affinity-purified polyclonal antibodies can be used.

In a two-antibody assay, one antibody is purified and bound to a solid phase. Any solid phase can be used; however, for most applications, a PVC microtiter plate is preferred. The bound antibody (to a well of a microtiter plate, for example) is unlabeled and is referred to as the “capture antibody.” The amount of antibody to be used will depend on the individual assay, but an amount of about 1 μg/well generally gives maximal binding. Higher or lower amounts of capture antibody can also be used. The wells can then be washed and sample added to the wells to allow the antigen (in this case, BPSA protease or BPSA aminopeptidase) in the test solution to bind to the solid phase. Unbound proteins can be removed by washing and a labeled second antibody can be added. Alternatively, the sample and the second labeled antibody can be added simultaneously. After washing, the assay can be quantitated by measuring the amount of labeled second antibody that is bound to the solid phase. A most preferred embodiment of the present invention utilizes a monoclonal antibody as the first unlabeled antibody and a monoclonal antibody as the second labeled antibody. The detection method used to quantitate the amount of bound labeled antibody depends on the label used. Antibodies can be labeled conveniently with iodine, enzymes, or biotin. Calorimetric or other detection methods can be used.

The BPSA protease or BPSA aminopeptidase of the present invention can be immobilized and used as “capture antigens” to bind and immobilize anti-BPSA-protease and anti-BPSA-aminopeptidase antibodies from a sample to be assayed for such antibodies. The bivalent complex of the enzyme and the antibodies is then detected, e.g., in the case of human physiological material, by reacting it with an anti-human IgG antibody which comprises a detectable label or a binding site for a detectable label. In the latter case, the binding site is itself reacted with a compound specific for the binding site, which itself comprises a detectable label. Useful detectable labels include enzymes, radio labels, or fluorescent labels. The resultant ternary or quaternary complex can then be detected and/or quantified via the detectable label, i.e., via an enzyme-substrate color-forming reaction, radio emission, agglomeration, and the like.

Alternatively, the BPSA protease (or BPSA aminopeptidase) can be labeled with a detectable label, such as via one or more radio labeled peptidyl residues, and can be used to compete with endogenous BPSA protease (or BPSA aminopeptidase) for binding to their respective antibodies, i.e., as a capture antigen to bind to antibodies in a sample of a physiological fluid via various competitive immunoassay formats.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Materials and Methods

### Isolation of PSA from Prostate Tissue

Prostate tissue was frozen in liquid nitrogen and pulverized to a fine powder in a metal tissue pulverizer maintained in liquid nitrogen. The frozen tissue powder (100-300 mg) was homogenized in 3 ml of phosphate buffered saline (PBS) containing a protease inhibitor cocktail (Complete, Boehringer Manheim, Indianapolis, Ind.) using a glass tissue homogenizer. The sample was then centrifuged to remove cell debris and the supernatant solution filtered through a 0.2 μm membrane.

PSA was purified from the filtered supernatant solution by passage over an immuno-affinity column containing bound anti-PSA mAb, PSM773 (Beckman Coulter, Inc., San Diego, Calif.) at 5 mg per ml of resin. PSM773 has been shown previously to be a PSA-specific mAb, and to have specificity for mature, cleaved, and precursor forms of PSA (28-30). The column was washed with 40 volumes of PBS containing 0.1% reduced Triton-X100, and the PSA eluted with 100 mM glycine pH 2.5, containing 200 mM sodium chloride. The eluant was immediately neutralized with 10% vol/vol 1M Tris pH 8.0.

### Preparation of BPSA from Seminal Plasma

Processed, filtered seminal plasma was diluted 1:20 in PBS and passed over an immunoaffinity column with bound anti-PSA mAb, PSM773. The column was washed with 20 volumes of PBS containing 0.1% reduced Triton X100, and the PSA eluted with 100 mM glycine pH 2.5 containing 200 mM sodium chloride.

### HIC-HPLC of PSA

High-performance hydrophobic interaction chromatography (HIC-HPLC) was performed using a polypropylaspartamide column (PolyLC, distributed by Western Analytical, Temecula, Calif.). The column was 4.6×250 mm in length with a 1000 Å pore size. Samples were applied in 1.5 M ammonium sulfate and eluted with a gradient. Buffers were prepared as follows. Buffer A consisted of 1.2 M sodium sulfate, 25 mM sodium phosphate, and a pH of 6.3, and Buffer B consisted of 50 mM sodium phosphate and 5% v/v 2-propanol. The gradient was 0-30% B for 1 minute, 30-60% B 10 minutes, followed by re-equilibration in Buffer A. High-sensitivity peak detection was obtained with a Varian Model 9070 (Varian Medical Systems, Palo Alto, Calif.) scanning fluorescence detector using an excitation of 232 nm and an emission of 334 nm to detect the tryptophan residues in protein.

### Amino Acid Sequencing of PSA

N-terminal sequence analysis of the samples was performed on a PE-Applied Biosystems Model 492 amino acid sequencer (PE-Applied Biosystems, Foster City, Calif.). Purified PSA and peaks collected by HIC-HPLC were applied directly to Prosorb cartridges (PE-Applied Biosystems, Foster City, Calif.), washed three times with 0.1 ml 0.01% trifluoroacetic acid, and applied to the sequencer.

### Characterization by SDS-PAGE

SDS-PAGE was performed using 4-20% gradient mini-gels (Invitrogen, Carlsbad, Calif.) under reducing or non-reducing conditions, as indicated. Samples were electroblotted onto nitrocellulose using standard procedures. Primary antibody PSM773 was used at 5 μg/ml and secondary antibody (Goat anti-mouse-HRP, 1:500, Jackson Immunoresearch Laboratories, Inc., West Grove, Pa.) was used to probe the blots. The immunoreactive signals were detected by ECL system (Amersham, Buckinghamshire, England) according to the manufacturer's instructions. SDS-PAGE samples for sequencing were electroblotted onto polyvinyldifluoride (PVDF) membranes (PE-Applied Biosystems) and the bands were sequenced directly on the PE-Applied Biosystems Model 492 amino acid sequencer.

### Incubation of PSA with Lysyl Endopeptidase C

Samples of PSA and proPSA were incubated with 0.1% lysyl endopeptidase C (Wako Cemicals USA, Richmond, Va.) in PBS at pH 7 for the times indicated in the figures. The LysC reaction was quenched by the addition of a 10-fold molar excess of aprotinin prior to analysis by HIC-HPLC. The internal cleavage sites of the different HIC-HPLC peaks were determined by N-terminal sequencing.

### Example 1

### Internal Cleavage Sites in PSA Digested with Lysyl Endopeptidase C (LysC)

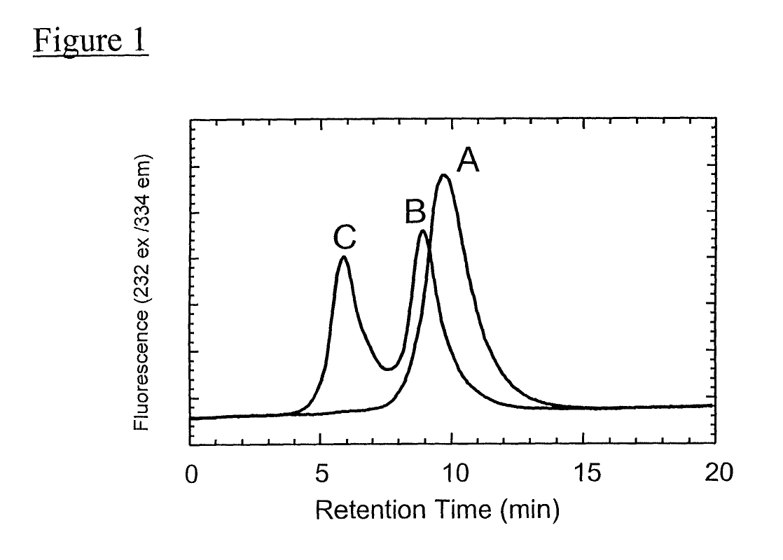

PSA was affinity purified from seminal plasma and further purified by HIC-HPLC to yield the single peak containing no BPSA (Peak A, FIG. 1). PSA of peak A contained approximately 35% cleavage at Lys145, and 5% cleavage at Lys182, which are typical values for seminal plasma PSA. After PSA was digested with LysC, there was no PSA intact or cleaved only at Lys145. Instead, two new peaks were observed (B and C). N-terminal sequencing revealed that peak B represented PSA entirely cleaved at Lys182 and peak C represented PSA cleaved at both Lys145 and Lys182 (so called, the BPSA form of PSA).

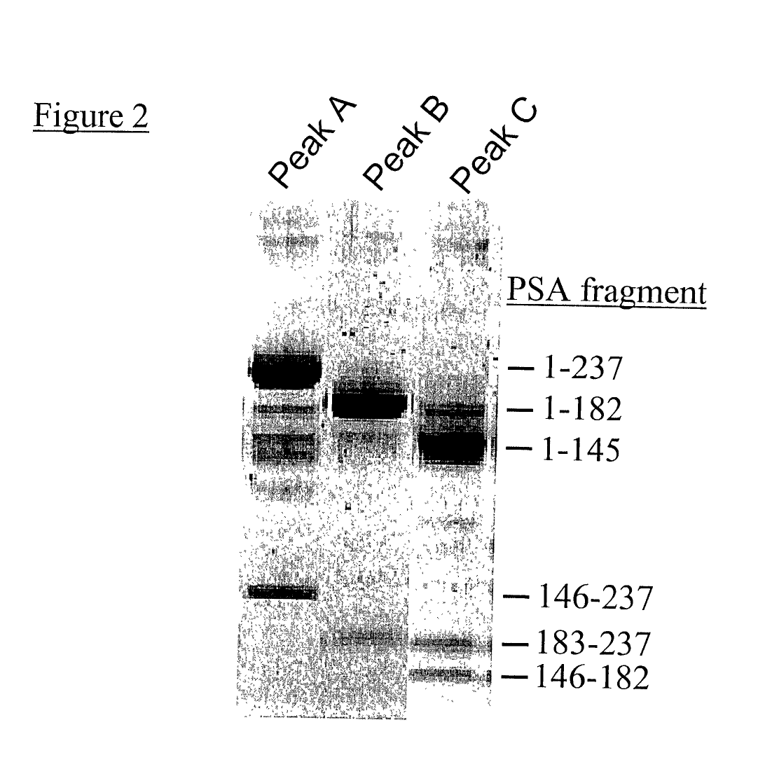

SDS-PAGE was carried out under standard reducing conditions with 2-mercaptoethanol, and the bands from Peaks A, B and C were confirmed by amino acid sequencing (FIG. 2). Lane 1 shows the purified PSA starting material, which is mostly intact PSA with a low level of internal peptide bond cleavages. The bands at 22 kDa and 10 kDa are the fragments containing residues 1-145 and 146-237, respectively, resulting from cleavage at Lys145. Lane 2 shows that Peak B contains no intact PSA, and almost entirely cleaved at Lys182. The fragments at 29 kDa and 6 kDa represent fragments containing residues 1-182, and 183-237, respectively. Lane 3 shows the fragment pattern of Peak C (BPSA). The main bands are found at 22 kDa, 6 kDa, and 4 kDa and represent fragments comprised of residues 1-145, 146-182, and 183-237, respectively.

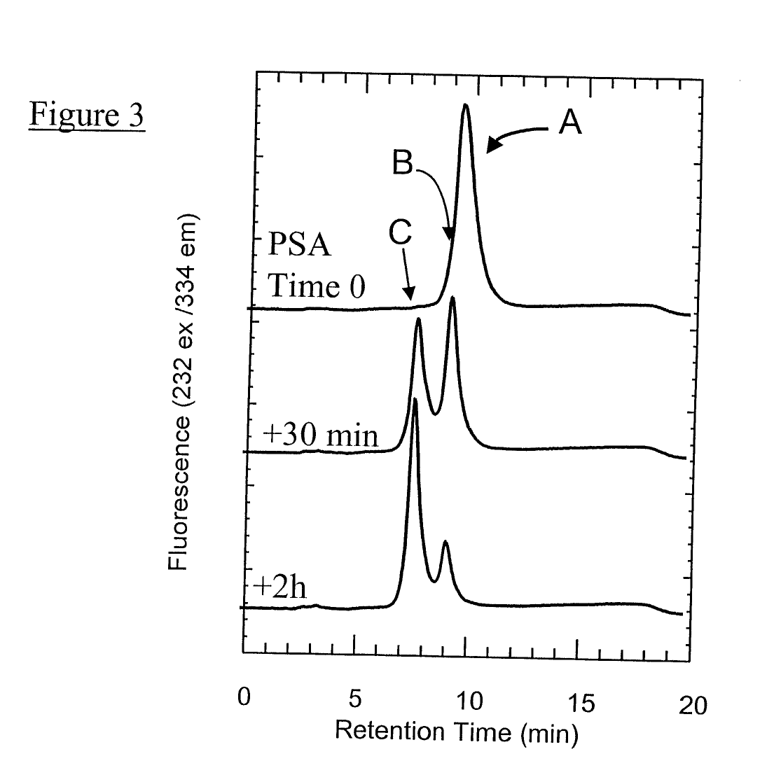

We also examined the digestion of PSA with LysC in vitro to determine the relative susceptibility of the Lys145 and Lys182 sites. The digestion reaction was incubated for 30 minutes or two hours and then was analyzed by HIC-HPLC, as shown in FIG. 3. FIG. 3 demonstrates the dynamics of the digestion from the initial cleavage at Lys182 at 30 minutes of incubation to the dual cleavages at Lys145 and Lys182 at 2 hours of incubation.

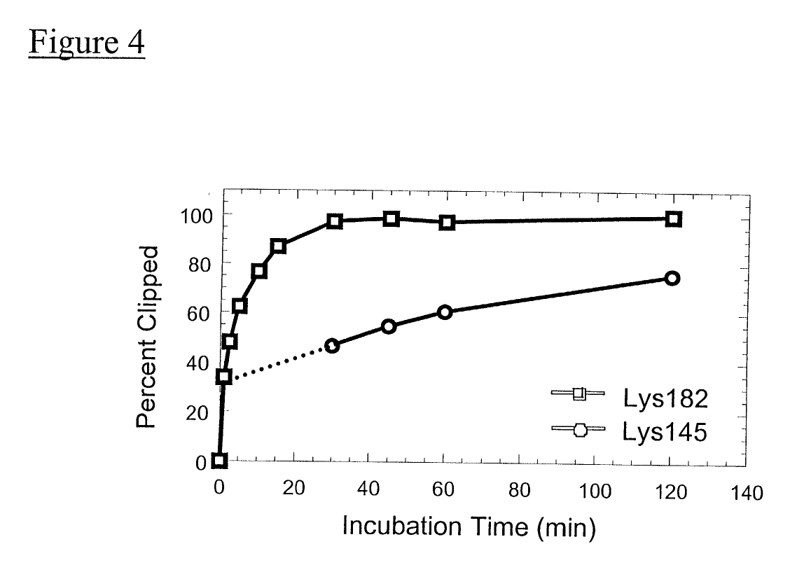

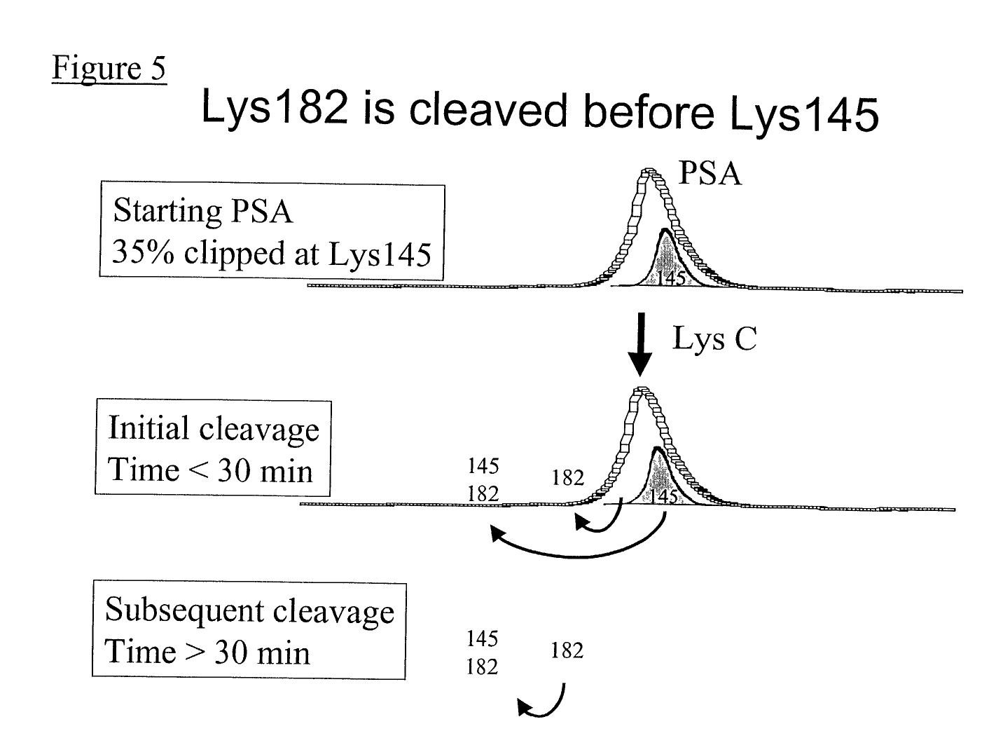

FIG. 4 shows the percent cleavage at Lys145 and Lys182 when incubated with LysC. The initial rate of cleavage at Lys182 was calculated to be 12% per minute, while cleavage at Lys145 was 0.3% per minute based on time points greater than 30 minutes. Use of the later time points for this calculation was necessary, since the %Lys145 cleavage was based on the appearance of the BPSA peak at 8 minutes. However, the starting PSA contained 35% cleavage at Lys145 and so the initial rapid cleavage at Lys182 resulted in artificial levels of BPSA arising from LysC cleavage at Lys182, and not from cleavage at Lys145. This is illustrated in FIG. 5. After 30 minutes of incubation, 100% of the PSA had been cleaved at Lys182 as indicated by the complete shift of the 10 minute PSA peak to the 9.5 minute Lys182-cleaved PSA position, and the 35% of the PSA endogenously cleaved at Lys145 which eluted as BPSA after cleavage at Lys182. Continued incubation resulted only in the much slower cleavage of the Lys182-cleaved PSA at the Lys145 site. It should be noted that the rate of Lys182 cleavage was the same for either the intact PSA or the PSA endogenously cleaved at Lys145, indicating that the Lys182 cleavage was not altered or affected by the presence or absence of the cleavage at Lys145.

ProPSA (pPSA) was also incubated with LysC to determine if there was any difference between the cleavage of the Lys145 and Lys182 sites due to the altered folding in the zymogen form of PSA. This is an important consideration since PSA is expressed as the zymogen form and converted extracellularly to active PSA. Presently, it is not known whether BPSA cleavages initially occur in the active or zymogen form of PSA in prostate tissues; it is possible that one or both sites are blocked in the zymogen. The cleavage of the Lys182 and Lys145 sites in pPSA was also monitored by HIC-HPLC.

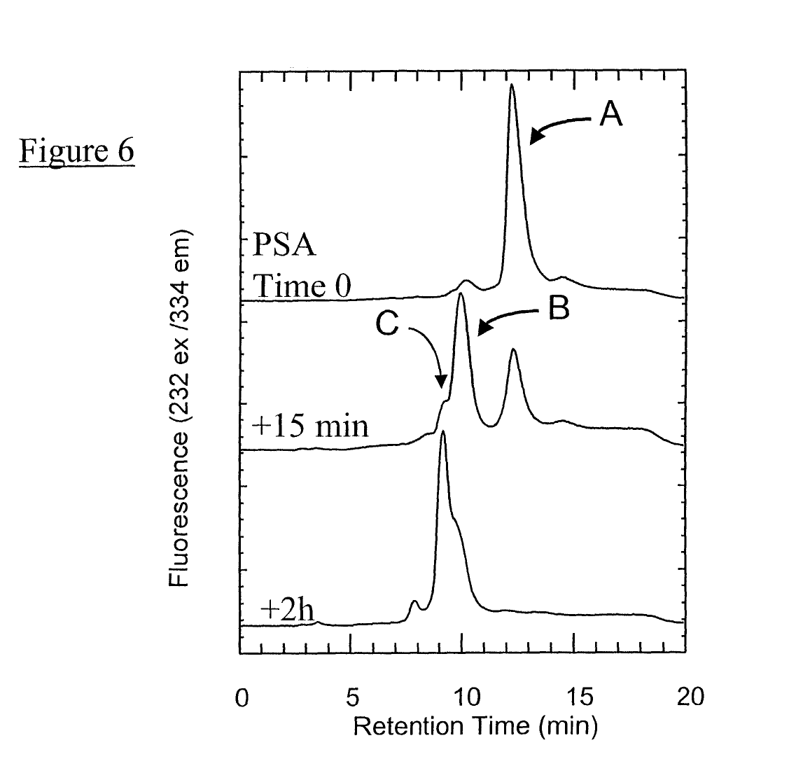

FIG. 6 shows the HIC-HPLC profile of selected time points during incubation. Time 0 profile was recorded before pPSA was incubated with LysC. Peak A indicates the elution of pPSA, which elutes by about 12 minutes, which is later than PSA. After 15 minutes of incubation with LysC (+15 min profile), the major peak at 10 minutes reflects pPSA internally cleaved at Lys182. Unlike the purified mature PSA, pPSA was isolated from a recombinant expression system and contains no endogenous internal peptide bond cleavages. After further incubation (+2 h profile), the major peak shifts from 10 minutes to 9.5 minutes, which reflects the additional cleavage at Lys145. Accordingly, the peak at 9.5 minutes contains both cleavages at Lys145 and Lys182.

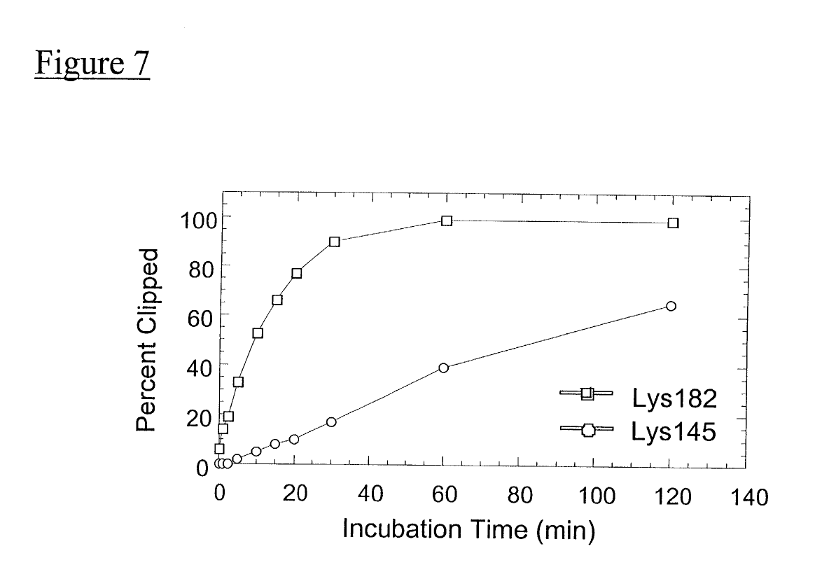

FIG. 7 shows the rate of cleavage at each site. The initial rate of cleavage of the Lys182 in pPSA was 8% per minute, similar, within experimental error, to the Lys182 cleavage of mature PSA. As with mature PSA, the cleavage at Lys145 is much slower than the Lys182 at about 0.6% per minute. These results demonstrate that both the mature and zymogen forms of PSA are similarly susceptible to cleavage at the Lys145 and Lys182 sites, and that the Lys182 cite is cleaved by far more rapidly in both active and zymogen PSA. It is therefore possible that the protease(s) responsible for the formation of BPSA could act on either the zymogen or active forms of PSA.

These results show that the Lys182 site is far more susceptible to cleavage than the Lys145 site. Earlier cleavage at Lys145 has no effect on the rate of cleavage at Lys182. The discovered preference and high rate of cleavage at the Lys182 site is contrary to the expectations of the art. The described result herein is particularly unexpected given the typical profile of internal cleavages in seminal plasma PSA where Lys145 is the predominant cleavage site.

### Discussion

One method of identifying the protease responsible for the formation of BPSA is to understand the cleavage of the Lys145 and Lys182 sites. Unexpectedly, we have found no significant cleavage of BPSA at Arg85 suggested as a PSA cleavage site by other research groups. This finding suggests that the protease of the present invention (referred to as BPSA protease) has a higher specificity for the lysine residues.

We have investigated the relative rate of cleavage of Lys145 and Lys182 by lysyl endopeptidase C (LysC) (see Example 1). Purified PSA treated with LysC was cleaved between Lys145 and Lys146, and between Lys182 and Ser183. Upon limited digestion, the cleavages at Lys145 and Lys182 were the only cleavage sites. The reaction was monitored by HIC-HPLC, as shown in FIG. 1.

We also examined the digestion of PSA with LysC in vitro to determine the relative susceptibility of the Lys145 and Lys182 sites to the enzymatic digestion. LysC, like trypsin, has a wide specificity for lysine residues, and is not considered very selective for the binding of protein substrates based on the amino acids surrounding the lysine residues. Thus, cleavage by LysC may be considered more dependent on the availability of the lysine residue based on protein folding as well as thermodynamic properties. We found that Lys182 is cleaved approximately 30 times more rapidly than Lys145 by LysC.

The precursor form of PSA (pPSA) was also incubated with LysC to determine whether there was any difference between the cleavage of the Lys145 and Lys182 sites due to the altered folding in the zymogen form of PSA (Example 1). This is an important consideration since PSA is expressed as the zymogen form and converted extracellularly to active PSA. In prostate tissues it is not known if BPSA cleavages initially occur in the active or zymogen form of PSA, and it is possible one or both sites are blocked in the zymogen. We discovered that pPSA is cleaved at rates that are compatible to the ones observed for BPSA and that the Lys182 site in PSA is cleaved more rapidly than the Lys145 site. Therefore, the BPSA protease responsible for the formation of BPSA may react with either the zymogen or active forms of PSA.

The discovered preference and high rate of cleavage at the Lys182 site is contrary to the expectations of the art. The result described herein is particularly unexpected given the typical profile of internal cleavage sites in seminal plasma PSA where Lys145 is the predominant cleavage site.

Because BPSA is elevated in hyperplastic tissues that contain BPH nodules, but not elevated in BPH tissues without nodules, the specific protease is present predominately in BPH tissues and, similarly to LysC, has a distinct preference for the Lys182 site.

### Example 2

### Lys145 and Lys182 Internal Cleavage Sites in BPH Isolated from Transition Zone Tissue

We measured the levels of internal cleavage sites at Lys182 and Lys145 in BPH transition zone tissue obtained by transurethral resection of the prostate (TURP). The TURP specimens contained variable levels of BPSA, as determined by HIC-HPLC, and were analyzed by amino acid sequencing for individual cleavages at Lys145 and Lys182. For the purposes of this example, the cleavage at Lys145 is defined as the combined cleavages at Lys145 as well as the cleavage at Lys146 (Lys146 cleavage is explained in Example 3). We analyzed six TURP tissues containing BPSA levels from 3% to 23% of the total PSA, as determined by HIC-HPLC. N-terminal sequence analysis showed that the overall cleavage of PSA at Lys182 was proportional to the amount of BPSA identified in the sample and ranged from 3% to 24% cleavage of PSA in these samples.

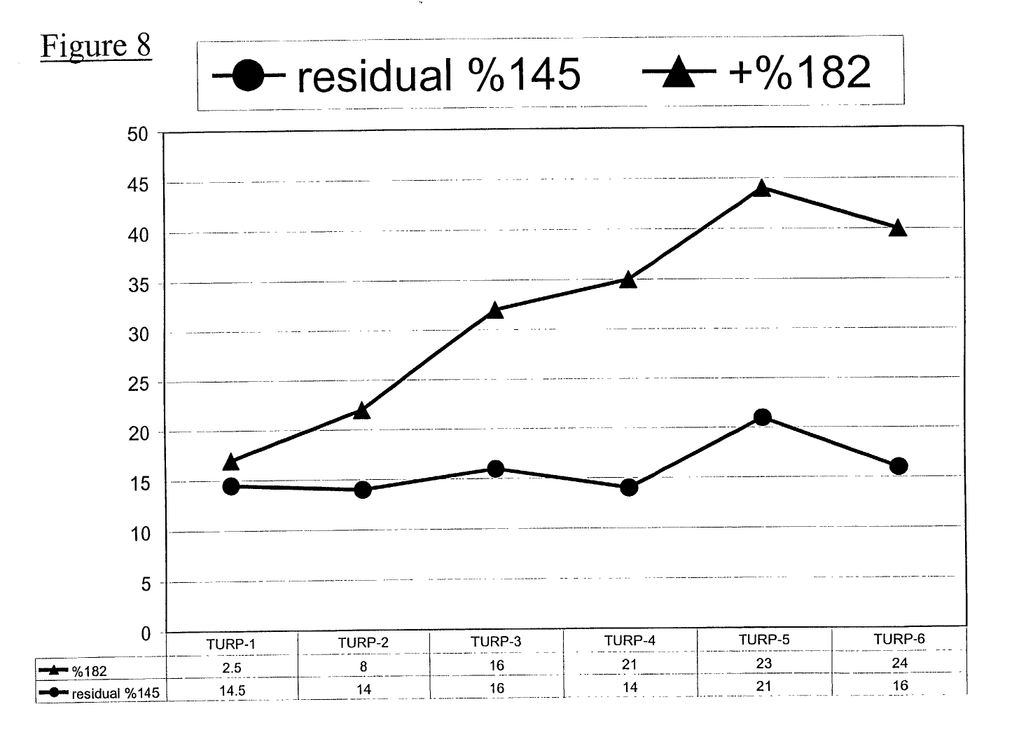

We found that the percentage of PSA cleaved at Lys145 increased proportionally with the presence of the Lys182 cleavage. This result is expected because the Lys145 cleavage is one of the major cleavage sites in BPSA. However, when the percent of PSA with Lys182 cleavage (determined by sequencing) was subtracted from the percent of PSA with Lys145 cleavage, the residual Lys145 not associated with BPSA (i.e., not associated with PSA cleaved at both Lys145 and Lys182) remained relatively constant. In FIG. 8, the percentage of PSA cleaved at Lys182 (%182) is compared to PSA with the “residual” Lys145 cleavage (residual %145) for each sample. Accordingly, the combined values of %182 and residual %145 represent the total percent of PSA cleaved at Lys145. Thus, the overall eight-fold increase of the level of PSA cleaved at Lys145 from TURP-1 to TURP-6 can be accounted for almost entirely by the eight-fold increase in the BPSA (PSA cleaved at both Lys145 and Lys182). On the other hand, the level of PSA cleaved only at Lys145 is about the same in all six TURP samples and is consistent with a background level of PSA cleaved at Lys145 that is present in the non-BPH tissue. This background level may represent the predominant Lys145 cleavage found in pooled seminal plasma from normal donors.

Aside from the PSA cleaved only at Lys145 (PSA-145), we also determined the levels of PSA cleaved only at Lys182 (PSA-182). Since PSA-145 did not vary as BPSA levels changed almost eight-fold, we investigated whether the level of PSA-182 correlates with the level of BPSA. We have previously shown that when BPSA is purified away from whole PSA, the remaining PSA, which elutes at 10 minutes by HIC-HPLC, still contains low levels of the Lys182 cleavage (22). We believe that this remaining PSA must be PSA-182 since in vitro studies described in Example 1 showed that an additional cleavage at Lys145 in the same PSA molecule leads to the formation of BPSA and results in a shift of HIC-HPLC retention time from 10 minutes to 8 minutes. While the majority of PSA cleaved at Lys182 is associated with BPSA (dual Lys145/Lys182 cleavages), we found that about 20% of the Lys182 is associated with PSA-182 (single Lys182 cleavage). BPSA and PSA-182 were purified from the TURP samples by immunoaffinity chromatography using the antibody mAb PS2E290, which has specificity for the Lys182 cleavage site.

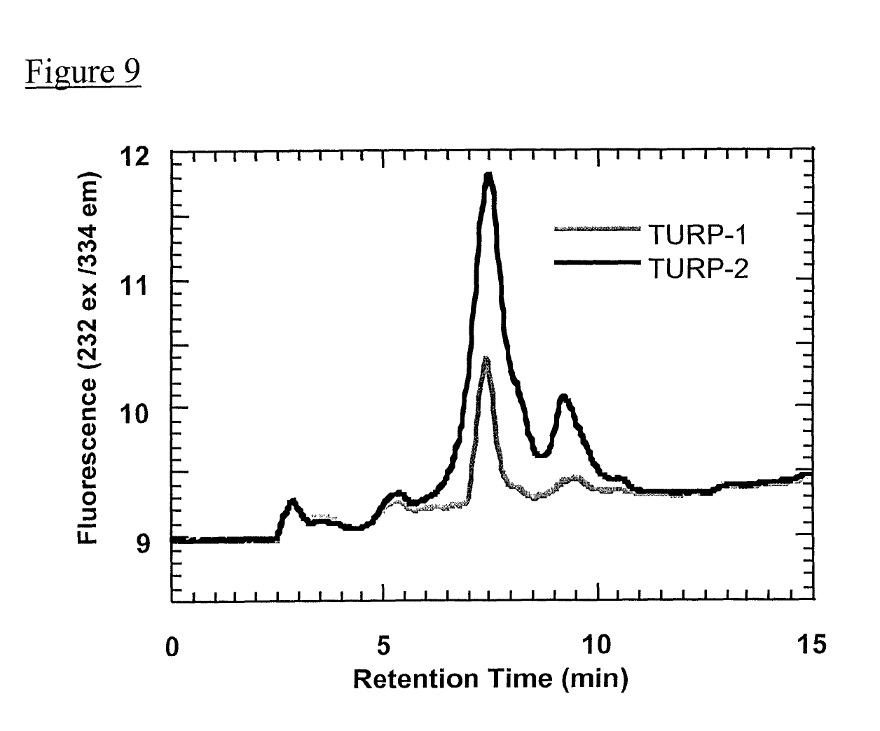

The eluant from this affinity column was subsequently resolved by HIC-HPLC into the BPSA and the PSA-182 components. The results for samples TURP-1 and TURP-6 are shown in FIG. 9. Sample TURP-1 contained the lowest levels of PSA with Lys182 cleavages (3%) and BPSA (4%), while TURP-6 contained the highest levels of PSA with Lys182 cleavages (24%) and BPSA (23%).

We found that while sample TURP-6 contained about eight times more BPSA than sample TURP-1, the relative amount of PSA-182 (PSA with a single cleavage at Lys182) as compared to the amount of BPSA in both samples was about the same (PSA-182 was about 21% of BPSA). Accordingly, the calculated amount of the total PSA cleaved only at Lys182 was 0.6% (21% of BPSA, which was 4% of total PSA) in TURP-1 and 4.8% (21% of BPSA, which was 23% of total PSA) in TURP-6. By contrast, PSA cleaved only at Lys145 was found to be 15% and 16% in TURP-1 and TURP-6, respectively (FIG. 8). Table 1 summarizes these findings.

TABLE 1

| | | % PSA-145 | % PSA-182 |
|---|---|---|---|
| | % BPSA by | (PSA cleaved at | (PSA cleaved at |
| Tissue | HIC-HPLC | Lys145 only) | Lys182 only) |
| TURP-1 | 3 | 15 | 0.6 |
| TURP-6 | 23 | 16 | 4.8 |

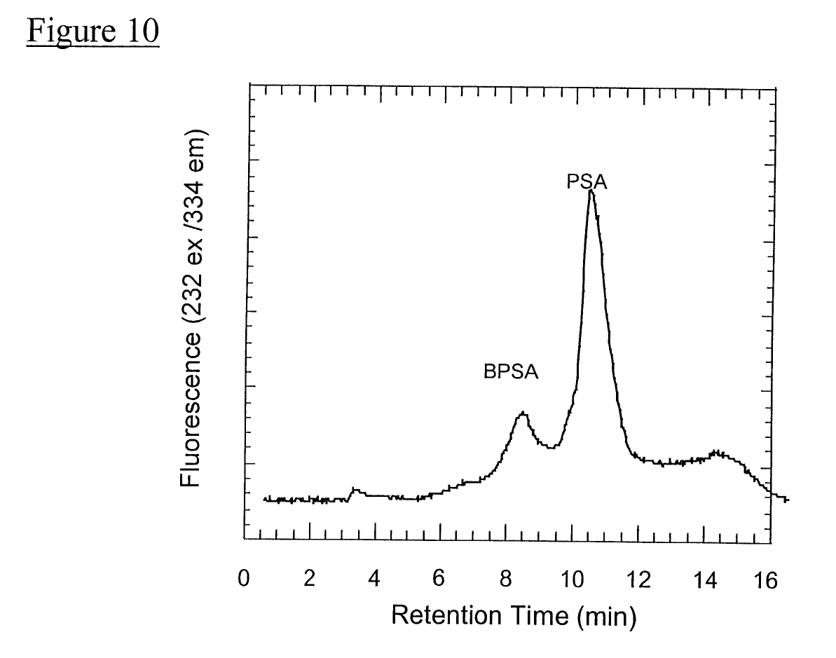

The percentage of PSA-182 in the TURP-6 sample was also confirmed directly by the N-terminal sequencing. PSA from the TURP-6 sample was resolved by HIC-HPLC, as shown in FIG. 10. The peak eluting at 10 minutes was collected and sequenced directly. N-terminal sequencing of the 10 minute peak showed 5% of Lys182 cleavage, in agreement with the calculated Lys182 cleavage (4.8%).

These results indicate that the same protease cleaves PSA at Lys182 to produce BPSA (dual Lys182/Lys145 cleavages) and PSA-182 (single Lys182 cleavage). Since the residual (non-BPSA associated) cleavage at Lys145 is not correlated to changes in BPSA concentration, this further indicated that the BPH tissue-associated protease, which cleaves PSA to form BPSA, has a preference for Lys182. This is in contrast to the protease that generates PSA with the more commonly found Lys145-only clip found in seminal plasma. The Lys145 protease activity is clearly not elevated in hyperplastic tissues as seen with the comparison of TURP-1 and TURP-6. The Lys145 cleavage is present in 30-40% of the seminal plasma PSA, even in young men with no BPH. Thus, the BPSA prostease is a distinct protease in hyperplastic prosate tissue. These results are consistent with the results of the in vitro experiments of Example 1.

### Example 3

### Additional Internal Cleavage Sites in BPSA isolated from Transition Zone Tissue and Seminal Plasma

We examined TURP tissue and seminal plasma samples for evidence that other internal PSA cleavage sites are associated with BPSA. Previously, we have shown that BPSA in seminal plasma is identical to the tissue BPSA (22), which is consistent with a hypothesis that seminal plasma BPSA is derived from the prostate tissue. Analysis of the six TURP specimens of Example 2 identified elevated level of cleavages between Ile1 and Val2 (Ile1 cleavage) and Lys146 and Leu147 (Lys146 cleavage) resulting in two novel N-termini of PSA beginning at Val2 and Leu147. Ile1 and Lys146 cleavages were particularly high in the samples with elevated Lys182/BPSA.

FIG. 11 shows the relative percentages of these three PSA-associated cleavage sites found in the TURP samples. Because both the Val2 and Leu147 N-termini resulted from the removal of a single amino acid from the Ile1 N-terminus and Lys146, respectively, it is hypothesized that an amino-terminal aminopeptidase is induced in the hyperplastic tissues. Accordingly, the Lys146 cleavage is a result of the aminopeptidase activity on Lys146 of the previously cleaved fragment between Lys145 and Lys146, and not due to altered proteolytic specificity of the trypsin-like endoprotease normally associated with the cleavage at Lys145. In the TURP tissue, the Ile1 and Lys146 cleavages were present in nearly 50% of BPSA and only in about 10% of other PSA forms. This result is consistent with the presence of the aminopeptidase in the hyperplastic tissues where the BPSA is being formed.

Similar results were obtained when PSA was purified from 18 individual seminal plasma specimens and subsequently subjected to N-terminal sequencing. The results were sorted by increasing levels of Lys182 and graphed in FIG. 12. FIG. 12 shows that no significantly elevated levels of Ile1 or Lys146 are found individually, but rather indicates that when one cleavage is elevated, the other one is also elevated at a similar level. This result also supports our conclusion that a single aminopeptidase is responsible for cleavages at both Ile1 and Lys146.

Both cleavages were observed predominantly in the samples with the highest levels of the Lys182 cleavage. Since Ile1 and Lys146 cleavages were not observed in PSA containing less than 10% BPSA, the expression of the aminopeptidase probably has a lower activity than the protease responsible for the cleavage at Lys182.

These results indicate the presence of an aminopeptidase in BPH tissues that is not normally present in non-nodular prostate tissues. This protease cleaves a single amino acid from the N-terminus and the amino-terminus of the previously cleaved protease between the Lys145 and Lys146 fragment. Thus, this amino peptidase has a specificity, which includes, but is not necessarily limited to, Isoleucine and Lysine residues. We did not observe Thr184 cleavages that would result from the removal of a single amino acid (Ser183) from the amino-terminus of Lys182 cleavage in BPSA. Since Lys182 is the preferred cleavage site in BPSA and since no Thr184 cleavages were observed, the aminopeptidase disclosed here does not have significant specificity for Serine residues.

The analysis of BPSA from both tissues and seminal plasma revealed trypsin-like protease activity resulting in cleavages at Lys145 and Lys182, and aminopeptidase activity resulting in Ile1 and Lys146 cleavages. Another cleavage that is typically present in seminal plasma PSA, Arg85, was not identified in samples of BPSA purified by HIC-HPLC from both tissues and seminal plasma. It is therefore evident that the proteases present in nodular BPH tissue do not have specificity for the Arg85 site.

### Example 4

### Method for the Isolation of the BPSA Protease

The methods and procedures outlined in Examples 1-3 may be used as screening protocols to identify the BPSA protease. Several systems known in the art may be used to identify and evaluate BPH proteases such as gene expression systems and BPH/prostate cell lines, but the preferred method would employ the analysis of BPH tissue extracts of BPH tissues that are known to contain elevated levels of BPSA. The preference for Lys182 cleavage site can be used as a screening tool to identify the BPSA protease. If the BPSA protease is capable of cleaving PSA at the Lys145 sites, it would be expected that the cleavage activity occurs much more slowly than the cleavage at Lys182, at least 2 to 10 times more slowly. Therefore, the conversion of PSA into BPSA (dual Lys145/182 cleavage) or to PSA with a single cleavage at Lys182 may be used to identify this protease in hyperplastic tissues. The BPSA protease would have a higher preference for Lys182 as compared to Lys145, and no appreciable activity on the Arg85 site that is commonly observed in non-BPSA forms of PSA.

The preferred substrate to be used for the identification of the BPSA protease is PSA containing no BPSA. BPSA may be removed from purified seminal plasma PSA by HIC-HPLC, as indicated in FIG. 1. This generates a population of PSA which is described as PSA_{BPSA(-)}(22). Though not essential for this process, the low residual levels of PSA containing the internal cleavage at Lys182-only may also be removed from PSA_{BPSA(-)} by passage over PS2E290, a monoclonal antibody that has specificity for the cleavage at Lys182. Passage of PSA_{BPSA(-)} over an affinity column containing bound PS2E290 has been used successfully by us and generates PSA completely devoid of any significant internal cleavage at Lys182 as determined by N-terminal sequencing. Alternately, whole purified seminal plasma PSA may be passed over a PS2E290 affinity column, which removes both BPSA and PSA containing the cleavage at Lys182 only, and results in a population of PSA that contains no internal cleavage at Lys182. For the ;present invention, this latter procedure is the preferred procedure for obtaining the substrate for the identification of the BPSA protease.

Once the suitable PSA substrate has been obtained, here designated PSA_{Lys182(-)}, it may be incubated with extracts from hyperplastic BPH tissue that are known to contain endogenously high levels of BPSA. Approximately 200 mgs of BPH tissue should be frozen in liquid nitrogen and pulverized to a fine powder before being homogenized in 1 ml PBS buffer in a glass tissue grinder. The extract should be filtered with 0.2 micron filter, and passed over an anti-PSA affinity column containing bound PSM773 in order to remove all forms of PSA from the extract. During the process of extracting the BPH tissue, the PBS buffer solution may be used alone, or the PBS may contain reagents, chemical and protease inhibitors to help stabilize, protect or enhance the activity of the BPSA protease. Many such compounds are known in the art of enzyme purification and may contain, but are not limited to, calcium, magnesium, EDTA, EGTA, and specific protease inhibitors such as aprotinin, leupeptin, calpain, alpha1-antitrypsin, and alpha1-antichymotrypsin. In addition, other standard and well-known buffers may be substituted for PBS during the extraction procedure in order to alter the pH of the extraction mixture from pH 4 to pH 10, in order to test the stabilizing or enhancing effects of different pHs in the purification of the BPSA protease.

Once the suitable extract of BPH tissue is obtained, it may be tested for the ability to preferentially cleave PSA_{lys182(-)} at the Lys182 site. Several screening procedures may be used to determine the proteolytic activity of the BPH tissue extract in cleaving PSA_{lys182(-)} at Lys182. One preferred procedure involves the following steps. Bind biotinylated PSM773 to a streptavidin-coated microtiter plate and then incubate 10 ug PSA_{lys182(-)} per well. The PSM773 binds the PSA_{lys182(-)} to the plate where it can act as a substrate. To each well, add 50 ul of BPH tissue extract and incubate for 1 hour at 37° C. The BPH tissue extract may be undiluted or diluted in buffer prior to adding to the microtiter well in order to control the rate of enzymatic activity. In addition, each sample of extract may contain the chemicals and protease inhibitors described above in order to stabilize or enhance the activity of the BPSA protease. Alternately, PSA_{lys182(-)} may be incubated with the BPH tissue extract in a test tube prior to addition to the microtiter plate containing the bound PSM773.

The presence of PSA cleaved at Lys182 is detected in each well by washing the extract from wells and adding PS2E290 to detect the PSA cleaved at Lys182. In the preferred method, a PS2E290-alkaline phosphatase conjugate is used to detect the presence of the cleavage at Lys182. The PS2E290-alkaline phosphatase conjugate is allowed to react in the microtiter plate well at room temperature for 2 hours. The plate is then washed and the PS2E290-alkaline phosphatase conjugate is allowed to react in the microtiter plate well at room temperature for 1 hour. The plate is then washed to remove unbound conjugate and 100 ul of Sigma 4MU-p solution (Sigma Chemical Co, St. Louis, Mo.) is added to each well and allowed to react at room temperature. After 1 hour, the plate is read on an EG&G Wallac Victor instrument. Alternately, Europium-labeled PS2E290 may be employed as the detection antibody and measured by time-resolved fluorescence.

The BPSA protease may be purified from the extract by protein purification procedures commonly known in the art, such as size-exclusion, ion-exchange or hydrophobic interaction chromatography. In the preferred method, the extract is applied to size exclusion chromatography and fractions collected and tested for the ability to cleave PSA at the Lys182 site using the microtiter plate screening procedure developed above. The fractions containing the highest levels of BPSA protease activity may then be further purified by a combination of anion, cation, and hydrophobic interaction chromatography procedures, with fractions again being screened for the peak of BPSA protease activity with the microtiter plate assay described above. The enrichment in the amount of BPSA protease activity compared to total protein at each step of the purification can therefore be calculated and used to ensure that the appropriate BPSA protease activity is being enriched. In each step above, the overall purity may also be monitored by analyzing each fraction by SDS-PAGE. Additional purification steps, commonly known to those in the art, may be employed if further purification of the BPSA protease is necessary. These include but are not limited to fractionation steps with ammonium sulfate and chromatography using reversed and normal phase chromatography.

The identification of the BPSA protease is obtained by procedures commonly known in the art, but which preferentially include N-terminal sequencing. Purified fractions of the BPSA protease from the above procedures my be sequenced directly, or a substantially pure band on an SDS-PAGE gel or Western blot may also be N-terminally sequenced. If the N-terminus of the protein is blocked, the protein may be trypsin digested and the fragments N-terminally sequenced as we have described for another protein (31). The amino acid sequence obtained by N-terminal sequencing can be used to search commonly available protein data bases in order to determine if the BPSA protease sequence has been previously reported. If the BPSA protease has not been previously described, then a complete N-terminal sequencing of its fragment peptides after trypsin digestion can be used to establish the complete amino acid sequence in order to determine homology with other known proteases. Mass spectroscopy may also be used to determine the absolute molecular mass which is compared to the mass calculated from the amino acid sequence.

### Example 5

### Method for the Isolation of the BPH Aminopeptidase

The procedures for isolation and identification of the BPH aminopeptidase are similar to those described in Example 4, except that samples must be screened for the presence of cleavages after Ile1 and Lys146 but not after Ser183. The preferred substrate to be used for the identification of the BPH aminopeptidase is synthetic BPSA which contains only the internal cleavages at Lys145 and Lys182. Synthetic or in vitro BPSA may be prepared from purified seminal plasma PSA by treatment with LysC as described in Examples 1-3, and purified as indicated in FIGS. 1-3.

Once the synthetic BPSA substrate has been obtained, it may be incubated with extracts from nodular BPH tissue that are known to contain high levels of endogenous BPSA. Approximately 200 mgs of BPH tissue should be frozen in liquid nitrogen and pulverized to a fine powder before being homogenized in 1 ml PBS buffer in a glass tissue grinder. The extract should be filtered with 0.2 micron filter, and passed over a anti-PSA affinity column containing bound PSM773 in order to remove all forms of PSA from the extract. During the process of extracting the BPH tissue the PBS buffer solution may be used alone, or the PBS may contain reagents, and chemical and protease inhibitors to help stabilize, protect or enhance the activity of the BPSA protease. Many such compounds are known in the art of enzyme purification and may contain, but are not limited to, calcium, magnesium, EDTA, EGTA, and specific protease inhibitors such as aprotinin, leupeptin, calpain, alpha1-antitrypsin, and alpha1-antichymotrypsin. In addition, other standard and well-known buffers may be substituted for PBS during the extraction procedure in order to alter the pH of the extraction mixture from pH 4 to pH 10, in order to test the stabilizing or enhancing effects of different pHs in the purification of the BPSA protease.

Once the suitable extract of BPH tissue is obtained, it may be tested for the ability to preferentially cleave synthetic BPSA after Ile1 and Lys146. The preferred screening procedure for the detection of the BPH aminopeptidase is to incubate 10 ug of synthetic BPSA with 50 ul of BPH tissue extract for 1 hour at 37° C. The extract is then applied to a small affinity column containing bound PSM773 in order to purify the PSA away from the other proteins in the extract. The PSA eluted from the affinity column is subjected to N-terminal sequencing to measure the extent of cleavage after Ile 1 and Lys 146.

The BPH aminopeptidase may be purified from the extract by protein purification procedures commonly known in the art such as size-exclusion, ion-exchange, or hydrophobic interaction chromatography. In the preferred method, the extract is applied to size exclusion chromatography and fractions collected and tested for the ability to cleave PSA at the Lys182 site using the microtiter plate screening procedure developed above. The fractions containing the highest levels of BPH aminopeptidase activity may then be further purified by a combination of anion, cation, and hydrophobic interaction chromatography procedures, with fractions again being screened for the peak of BPH aminopeptidase activity with the microtiter plate assay described above. The enrichment in the amount of BPSA protease activity compared to total protein at each step of the purification can therefore be calculated, and used to ensure that the appropriate BPH aminopeptidase activity is being enriched. In each step above, the overall purify may also be monitored by analyzing each fraction by SDS-PAGE. Additional purification steps, commonly known to those in the art, may be employed if further purification of the BPH aminopeptidase is necessary. These include, but are not limited, to fractionation steps with ammonium sulfate and chromatography using reversed and normal phase chromatography.

The identification of the BPH aminopeptidase is obtained by procedures commonly known in the art, but which preferentially include N-terminal sequencing. Purified fractions from the above procedures my be sequenced directly, or a substantially pure band on an SDS-PAGE gel or Western blot may also be N-terminally sequenced. If the N-terminus of the protein is blocked, the protein may be digested and the fragments N-terminally sequenced as we have described for another protein (31). The amino acid sequence obtained by N-terminal sequencing can be used to search commonly available protein data bases in order to determine if the BPH aminopeptidase sequence has been previously described. If the BPSA has not been previously described, then a complete N-terminal sequencing of its fragment peptides after trypsin digestion can be used to establish the complete amino acid sequence in order to determine homology with other known proteases. Mass spectroscopy may also be used to determine the absolute molecular mass which is compared to the mass calculated from the amino acid sequence.

### Example 6

### Methods for Determination of Inhibitors of the BPSA Protease and BPH Aminopeptidase

Inhibitors of BPSA protease or BPH aminopeptidase may be identified using either the purified proteins, or fractions of BPH tissue extracts that contain partially or substantially pure protease proteins. Using the screening procedures described in Examples 4 and 5, the substantially purified proteins or BPH tissue extracts containing protease activity may be incubated with the appropriate substrate in each case, both with and without the presence of the compounds being tested for inhibitor activity. The same procedures employed to determine the hydrolytic activity in Examples 4 and 5 may therefore be used to screen for inhibitory activity of the compounds being tested. Thus, the development of inhibitory compounds is dependant on the assay and screening procedures described in Examples 4 and 5. Multiple techniques have been used for the development of inhibitors to enzymes and proteases, and are commonly known in the art. For example, the protocols are well known in the art for the development of inhibitors of the HIV protease (25) and the inhibitor of 5-alpha-reductase for the treatment of BPH (26,27). It is the unique aspect of the present invention that we have developed screening tools in order to test for the inhibition of BPSA protease and BPH aminopeptidase.

### Example 7

### Using Antibodies to Lys182 Protease and Aminopeptidase as Markers of BPH Nodules and Hyperplastic Cells and Tissues

The present invention may be embodied in other specific forms without departing from its essential characteristics. The described embodiment is to be considered in all respects only as illustrative and not as restrictive. The scope of the invention is, therefore, indicated by the appended claims rather than by the foregoing description. All changes which come within the meaning and range of the equivalence of the claims are to be embraced within their scope.

### Reference List

1. Catalona, W. J., Smith, D. S., Ratliff, T. L., Dodds, K. M., Coplen, D. E., Yuan, J. J., Tetros, J. A., and Andriole, G. L. Measurement of prostate-specific antigen in serum as a screening test for prostate cancer. N Engl J Med, 324: 1156-1161, 1991.

2. Oesterling, J. E. Prostate-specific antigen: a critical assessment of the most useful tumor marker for adenocarcinoma of the prostate. J Urol, 145: 907-923, 1991.

3. Labrie, F., Dupont, A., Suburu, R., Cusan, L., Tremblay, M., Gomez, J. L., and Emond, J. Serum prostate specific antigen as pre-screening test for prostate cancer. J Urol, 147: 846-851, 1992.

4. Rittenhouse, H. G., Finlay, J. A., Mikolajczyk, S. D., and Partin, A. W. Human kallikrein 2 (hK2) and prostate-specific antigen (PSA): Two closely related, but distinct, kallikreins in the prostate. Crit Rev Clin Lab Sci, 35: 275-368, 1998.

5. Watt, K. W. K., Lee, P. J., M'Timkulu, T., Chan, W. P., and Loor, R. Human prostate-specific antigen: Structural and functional similarity with serine proteases. Proc Natl Acad Sci USA, 83: 3166-3170, 1986.

6. Belanger, A., van Halbeek, H., Graves, H. C. B., Grandbois, K., Stamey, T., Huang, L. H., Poppe, I., and Labrie, F. “Molecular mass and carbohydrate structure of prostate specific antigen: studies for establishment of an international PSA Standard.” Prostate, 27: 187-197, 1995.

7. Catalona, W. J., Partin, A. W., Slawin, K. M., Brawer, M. K., Flanigan, R. C., Patel, A., Richie, J. P., deKermion, J. B., Walsh, P. C., Scardino, P. T., Lange, P. H., Subong, E. N., Parson, R. E., Gasior, G. H., Loveland, K. G., and Southwick, P. C. “Use of the percentage of free prostate-specific antigen to enhance differentiation of prostate cancer from benign prostatic disease: a prospective multicenter clinical trial.” JAMA, 279: 1542-1547, 1998.

8. Woodrum, D. L., Brawer, M. K., Partin, A. W., Catalona, W. J., and Southwick, P. C. “Interpretation of free prostate specific antigen clinical research studies for the detection of prostate cancer.” J Urol, 159: 5-12, 1998.

9. Lilja, H., Christensson, A., Dahlen, U., Matikainen, M. T., Nilsson, O., Pettersson, K., and Lovgren, T. “Prostate-Specific Antigen in Serum Occurs Predominantly in Complex with α₁-antichymotrypsin.” Clin Chem, 37: 1618-1625, 1991.

10. Stenman, U. H., Leinonen, J., Alfthan, H., Rannikko, S., Tuhkanen, K., and Alfthan, O. “A complex between prostate specific antigen and α₁-antichymotrypsin is the major form of prostate-specific antigen in serum of patients with prostatic cancer: assay of the complex improves clinical sensitivity for cancer.” Cancer Res., 51: 222-226, 1991.

11. Catalona, W. J. “Clinical utility of measurements of free and total prostate-specific antigen (PSA): a review.” Prostate, Supplement 7: 64-69, 1996.

12. Watt, K. W. K., Lee, P. J., M'Timkulu, T., Chan, W. P., and Loor, R. “Human prostate-specific antigen: structural and functional similarity with serine proteases.” Proc Natl Acad Sci USA, 83: 3166-3170, 1986.

13. Zhang, W. M., Leinonen, J., Kalkkinen, N., Dowell, B., and Stenman, U. H. “Purification and characterization of different molecular forms of prostate-specific antigen in human seminal fluid.” Clin Chem, 41: 1567-1573, 1995.

14. Price, H., McNeil, J. E., and Stamey, T. A. “Evolving patterns of tissue composition in benign prostatic hyperplasia as a function of specimen size.” Hum Pathol, 21: 578-585, 1990.

15. McNeal, J. “Pathology of benign prostatic hyperplasia. Insight into etiology.” Urol Clin North Am, 17: 477-486, 1990.

16. McNeal, J. E. “Origin and evolution of benign prostatic enlargement.” Invest Urol, 15: 340-345, 1978.

17. Chen, Z., Chen, H., and Stamey, T. A. “Prostate specific antigen in benign prostatic hyperplasia: purification and characterization.” J Urol, 157: 2166-2170, 1997.

18. Achbarou, A., Kaiser, S., Tremblay, G., Ste-Marie, L. G., Brodt, P., Goltzman, D., and Rabbani, S. A. “Urokinase overproduction results in increased skeletal metastasis by prostate cancer cells in vivo.” Cancer Res, 54: 2372-2377, 1994.

19. Bruckner, A., Filderman, A. E., Kerchheimer, J. C., Binder, B. R., and Remold, H. G. “Endogenous receptor-bound urokinase mediates tissue invasion of the human lung carinoma cell lines A549 and Calu-1.” Cancer Res, 52: 3043-3047, 1992.

20. Marks, L. S., Partin, A. W., Gormley, G. J., Dorey, F. J., Shery, E. D., Garris, J. B., Subong, E. N., Stoner, E., and deKemion, J. B. “Prostate tissue composition and response to finasteride in men with symptomatic benign prostatic hyperplasia.” J Urol., 157: 2171-2178, 1997.

21. Mikolajczyk, S. D., Millar, L. S., Wang, T. J., Rittenhouse, H. G., Wolfert, R. L., Marks, L. S., Song, W., Wheeler, T. M., and Slawin, K. M. “BPSA, a specific molecular form of free prostate-specific antigen, is found predominantly in the transition zone of patients with nodular benign prostatic hyperplasia.” Urology, 55: 41-45, 2000.

22. Mikolajczyk, S. D., Millar, L. S., Marker, K. M., Wang, T. J., Rittenhouse, H. G., Marks, L. S., and Slawin, K. M. “Seminal Plasma Contains ‘BPSA’, a Molecular Form of Prostate Specific Antigen that is Associated with Benign Prostatic Hyperplasia.” Prostate, In Press, 2000.

23. Bostwick, D. G. and Dundore, P. A. Biopsy Pathology of the Prostate. Chapman & Hall, 1997.

24. Mikolajczyk, S. D., Millar, L. S., Marker, K. M., Grauer, L. S., Goel, A. S., Cass, M. M. J., Kumar, A., and Saedi, M. S. “Ala217 is important for the catalytic function and autoactivation of prostate-specific human kallikrein 2.”Eur.J.Biochem., 246: 440-446, 1997.

25. Baker, C. T., Salituro, F. G., Court, J. J., Deininger, D. D., Kim, E. E., Li, B., Novak, P. M., Rao, B. G., Pazhanisamy, S., Schairer, W. C., and Tung, R. D. “Design, synthesis, and conformational analysis of a novel series of HIV protease inhibitors.” Bioorg.Med.Chem.Lett., 8: 3631-3636, 1998.

26. Stoner, E. “The clinical development of a 5 alpha-reductase inhibitor, finasteride.” J.Steroid Biochem.Mol.Biol., 37: 375-378, 1990.

27. Guarna, A., Danza, G., Bartolucci, G., Marrucci, A., Dini, S., and Serio, M. “Synthesis of 5,6,6-[2H3]finasteride and quantitative determination of finasteride in human plasma at picogram level by an isotope-dilution mass spectrometric method.”J.Chromatogr.B Biomed.Appl., 674: 197-204, 1995.

28. Wang, T. J., Linton, H. J., Sokoloff, R. L., Grauer, L. S., Rittenhouse, H. G., and Wolfert, R. L. “Antibody specificities for PSA and PSA fragments by SDS-PAGE Western blot analysis.” Tumor Biology, 20: 75-78, 1997.

29. Finlay, J. A., Day, J. R., and Rittenhouse, H. G. “Polyclonal and monoclonal antibodies to prostate-specific antigen can cross-react with human kallikrein 2 and human kallikrein 1.” Urology, 53: 746-751, 1999.

30. Kumar, A., Mikolajczyk, S. D., Goel, A. S., Millar, L. S., and Saedi, M. S. “Expression of pro form of Prostate-specific antigen by mammalian cells and its conversion to mature, active form by human kallikrein 2.” Cancer Res, 57: 3111-3114, 1997.

31. Mikolajczyk, S. D., Millar, L. S., Marker, K. M., Rittenhouse, H. G., Wolfert, R. L., Marks, L. S., Charlesworth, M. C., and Tindall, D. J. “Identification of a novel complex between human kallikrein 2 and protease inhibitor-6 in prostate cancer tissue.” Cancer Res, 59: 3927-3930, 1999.

What is claimed is:

1. A method for identification and isolation of a benign prostate specific antigen (BPSA) protease, the method comprising:
(a) providing an extract from a source with elevated BPSA;
(b) screening the extract for a BPSA protease that is capable of cleaving PSA of SEQ ID NO:1 at residue Lys182 but not at residue Arg85; and
(c) isolating the BPSA protease from the extract if it is present.

2. The method of claim 1, wherein the screening step comprises:
(a) contacting a sample from the extract with PSA under conditions sufficient to allow cleavage of PSA by the BPSA protease to form BPSA; and
(b) detecting the presence of BPSA, wherein the presence of BPSA indicates the presence of the BPSA protease.

3. The method of claim 1, wherein the protease additionally cleaves PSA at residue Lys145.

4. The method of claim 3, wherein the BPSA protease preferentially cleaves at Lys182 over Lys145.

5. The method of claim 4, wherein the BPSA protease cleaves Lys182 site about 30 times more rapidly than Lys 145 site.

6. The method of claim 1, further comprising a step of characterizing the isolated BPSA protease.

7. The method of claim 1, wherein the PSA extract is obtained from benign prostate hyperplastic tissues containing BPH nodules.

8. The method of claim 1, wherein the PSA extract is obtained from hyperplastic prostate cells.

9. A BPSA protease isolated by the method of claim 1.

10. A method for identification and isolation of a BPSA aminopeptidase, comprising:
(a) providing an extract from a source with elevated BPSA;
(b) screening the extract for a BPSA aminopeptidase that is capable of cleaving BPSA between residues Ile1 and Val2, and between residues Lys146 and Leu147; and
(c) isolating the BPSA aminopeptidase from the extract if it is present.

11. The method of claim 10, wherein the screening step comprises:
(a) contacting a sample from the extract with BPSA under conditions sufficient to allow cleavage of BPSA by the BPSA aminopeptidase to cleave BPSA between residues Ile1 and Val2, and between residues Lys146 and Leu147 to produce peptides of SEQ ID NO:2 and SEQ ID NO:3; and
(b) detecting the presence of the peptides of SEQ ID NO:2 and SEQ ID NO:3 wherein the presence of said peptides indicates the presence of the BPSA aminopeptidase.

12. The method of claim 10, wherein the BPSA aminopeptidase does not cleave BPSA at Ser183.

13. A BPSA aminopeptidase isolated by the method of claim 10.

14. A method for detecting the presence of a BPSA protease in a sample, the method comprising:
(a) providing a sample suspected of containing the BPSA protease;
(b) contacting the sample with PSA under conditions sufficient to allow cleavage of PSA by the BPSA protease to form BPSA; and
(c) detecting the presence of BPSA, wherein the presence of BPSA indicates the presence of the BPSA protease.

15. The method of claim 14, wherein the sample is a tissue, tissue secretions, or tissue extract obtain from hyperplastic tissues containing BPH nodules.

16. The method of claim 14, wherein the detecting step includes determining preferential cleavage of PSA at Lys182 as compared to Lys145.

17. A method of detecting the presence of a BPSA aminopeptidase in a sample, the method comprising:
(a) providing a sample suspected of containing the BPSA aminopeptidase;
(b) contacting the sample with BPSA under conditions sufficient to allow cleavage of BPSA by the BPSA aminopeptidase to cleave BPSA between residues Ile1 and Val2, and between residues Lys146 and Leu147 to produce peptides of SEQ ID NO:2 and SEQ ID NO:3; and
(c) detecting the presence of the peptides of SEQ ID NO:2 and SEQ ID NO:3 wherein the presence of said peptides indicates the presence of the BPSA aminopeptidase.

18. The method of claim 17, wherein the sample is a tissue, tissue secretions, or tissue extract obtained from hyperplastic prostate tissue containing BPH nodules.


SEQUENCE LISTING (5 sequences)

SEQ ID NO: 1 - protein, 237 aa, Homo sapiens
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 2 - protein, 144 aa, Homo sapiens
VGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPK

SEQ ID NO: 3 - protein, 36 aa, Homo sapiens
LQCVDLHVISNDVCAQVHPQKVTKFMLCAGRWTGGK

SEQ ID NO: 4 - protein, 7 aa, Homo sapiens
APLILSR

SEQ ID NO: 5 - protein, 54 aa, Homo sapiens
TCSGDSGGPLVCNGVLQGITSWGSEPCALPERPSLYTKVVHYRKWIKDTIVANP